(19) United States
(12) Patent Application Publication
(10) Pub. No.: US 2024/0024219 A1
(43) Pub. Date: Jan. 25, 2024

(54) Oral Care Compositions with a Natural Sweetener System

(71) Applicant: Colgate-Palmolive Company, New York, NY (US)

(72) Inventors: Michelle MIGUELINO, Somerset, NJ (US); Fusong SUN, Martinsville, NJ (US); Zhigang HAO, Bridgewater, NJ (US)

(73) Assignee: Colgate-Palmolive Company, New York, NY (US)

(21) Appl. No.: 18/256,515

(22) PCT Filed: Dec. 9, 2021
(86) PCT No.: PCT/US2021/062623

§ 371 Date: Jun. 8, 2023

Related U.S. Application Data
(60) Provisional application No. 63/122,964, filed on Dec. 9, 2020.

(51) Int. Cl. A61K 8/60; A61Q 11/00; A61K 8/44; A61K 8/27

(52) U.S. Cl. CPC A61K 8/602; A61Q 11/00; A61K 8/60; A61K 8/44; A61K 8/27; A61K 2800/5922

(57) ABSTRACT

The disclosure provides a natural sweetener system and oral care compositions comprising the same. In one aspect the natural sweetener system comprises a combination of a Rebaudioside A extract and enzymatically glucosylated steviol glycosides extract obtained from Stevia Rebaudiana Bertoni. The natural sweetener system of the disclosure unexpectedly improves the taste of oral care compositions masking the bitterness and metallic notes of Stevia Rebaudiana Bertoni.

### FIELD OF THE INVENTION

The present disclosure provides a natural sweetener system and describes oral care compositions comprising the natural sweetener system. In one aspect the natural sweetener system comprises a combination of Rebaudioside A and enzymatically glucosylated steviol glycoside extract obtained from Stevia rebaudiana Bertoni. The disclosure also describes methods of use and manufacture.

### BACKGROUND OF THE INVENTION

Stevia rebaudiana Bertoni plant is a known natural sweetener and can be used in oral care compositions because it does not contain any fermentable carbohydrates. However, one disadvantage is that the sweetener is believed to have negative taste attributes such as bitterness and metallic notes, when used at the levels required for sweetening oral care compositions. Therefore, its use is often limited to low levels.

Sweeteners can be used in oral care compositions, e.g., toothpastes, in order to increase consumer acceptance, palatability, which in turn increases the likelihood that the oral composition is used consistently and regularly.

Specific flavors have been found to appeal to certain groups of the population. Some consumers prefer a sweet combination of flavors. For example, many oral care compositions are sweet because they contain an artificial sweetener such as saccharin or xylitol. Stevia extracts have also been considered as sweeteners because they have much higher perceived “sweetness”, as compared to sugar, without the downside of contributing to tooth decay. However, some consumers have found stevia sugar substitutes, such as rebaudioside A, to have an unpleasant and lingering after-taste which can be characterized as bitter and metallic. Accordingly, incorporating stevia extracts into oral care compositions has been challenging.

Rebaudioside-A is believed to be the second most abundant sweet diterpene glycoside (1-3%) present in the leaves of Stevia rebaudiana Bertoni. However, in oral care compositions, the use of low purity (≤95%) rebaudioside A or high purity (≥95%) full spectrum stevia extract can result in unacceptable bitterness. Unbalanced lingering sweetness may also result from high purity (≥95%) rebaudioside-A. Consequently, there is a need for a sweeting system with a balanced flavor profile, and high consumer acceptance, that can be incorporated within oral care compositions.

### SUMMARY OF THE INVENTION

In one aspect, the disclosure describes an all-natural sweetener system for use in oral care compositions. Rebaudioside-A is a natural alternative to sucrose and has been estimated to be around 200-times sweeter than sucrose. However, rebaudioside A has been reported to have unpleasant and lingering after-taste which can be characterized as bitter and metallic.

However, in at least one aspect, the inventors have surprisingly discovered that a combination of rebaudioside A and one or more enzymatically glucosylated steviol glycosides extracts, can provide pleasant organoleptic properties and greatly reduce the negative taste attributes of rebaudioside A when the sweetener is incorporated within an oral care composition. In one aspect, the enzymatically glucosylated steviol glycoside extract is produced from Stevia rebaudiana leaves in the presence of an enzyme. In one aspect, the enzyme be selected from: β-cyclodextrin glucanotransferase (β-CGtase) (e.g., produced from an alkalophilic strain of Bacillus firmus), alpha-amylase enzyme (e.g., derived from Bacillus licheniformis), cyclomaltodextrin glucanotransferase (CGTase) (e.g., enzyme derived from Geobacillus stearothermophilus) and combinations thereof.

The enzymatically Glucosylated Steviol Glycosides extract is available as an extract comprising 80% by weight to 95% by weight of enzymatically Glucosylated Steviol Glycosides. The rest of the extract up to 100% comprises steviosides and rebaudioside which didn't react with the enzyme.

The natural sweetener system described herein may be used as sole sweetener system in oral care compositions or may be used in conjunction with other natural sweetener systems to give a cleaner and more balanced sweetness profile.

In one embodiment, there is provided an oral care composition comprising a natural sweetener system comprising Reb A in a purity of from 95% to 99%.

In another embodiment, there is provided an oral care composition comprising a natural sweetener system comprising enzymatically Glucosylated Steviol Glycosides extract.

In another embodiment, there is provided an oral care composition comprising a natural sweetener system comprising Reb A and enzymatically Glucosylated Steviol Glycosides extract.

In another embodiment, there is provided an oral care composition comprising a natural sweetener system comprising Reb A in a purity of from 95% to 99% and enzymatically Glucosylated Steviol Glycosides extract wherein the enzymatically Glucosylated Steviol Glycosides are in 80% weight to 98% weight, relative to the extract.

In another embodiment, there is provided an oral care composition comprising a natural sweetener system comprising Reb A in a purity of from 95% to 99% and enzymatically Glucosylated Steviol Glycosides extract wherein the enzymatically Glucosylated Steviol Glycosides are in 80% weight to 98% weight, relative to the extract, and an orally acceptable carrier.

In some embodiments, the oral care composition is in the form of a toothpaste, a dentifrice, a mouthwash, a mouth rinse, mouth spray, a topical oral gel or a denture cleanser. In another embodiment, the oral care composition is in the form of dental strips, beads, varnish, tablets, toothpaste tape, toothpaste film, toothpaste ribbon, dental floss, chewing gum, gummies, lozenges or toothpowder.

Optionally, the oral care compositions of the invention further comprise abrasives, stain prevention compounds, whitening agents, pigments, erosion prevention agents, opacifiers, antitartar compounds, polymer complexes, surfactants, humectants and/or combinations thereof. Optionally, the oral care compositions of the invention further comprise diluents, bicarbonate salts, pH modifying agents, foam modulators, thickening agents, flavoring agents, antibacterial agents, desensitizing agents, anticariogenic agents, fluoride ion sources, anticalculus agents, and/or combinations thereof. Optionally, the oral care composition comprises water. Optionally, the oral care compositions of the invention comprise cleaning agents, flavoring agents, sweetening agents, adhesion agents, surfactants, foam modulators, pH modifying agents, humectants, moisturizers, mouth feel agents, colorants, abrasives, preservatives, fluoride ion source, saliva stimulating agents, emollients, viscosity modifiers, diluents, emulsifiers, nutrients and/or combinations thereof.

In another embodiment, there is provided a natural sweetener system comprising Rebaudioside A and an enzymatically Glucosylated Steviol Glycosides extract; wherein Rebaudioside A has a purity of from 95% to 99%; and the enzymatically Glucosylated Steviol Glycosides extract has a content in enzymatically Glucosylated Steviol Glycosides from 80% by weight to 98% by weight relative to the dry weight of the extract. In a further embodiment, the natural sweetener system according to the invention comprises a ratio of Rebaudioside A to enzymatically Glucosylated Steviol Glycosides extract of 1:1, or 1:2, or 1:3, or 1:4, or 1:5, or 2:1, or 3:1, or 4:1, or 5:1, respectively. The natural sweetener system, according to the invention, is comprised from about 0.05% to about 3.00% weight, based on the total weight of the composition.

In another embodiment, there is provided an oral care composition comprising a natural sweetener system comprising Rebaudioside A and an enzymatically Glucosylated Steviol Glycosides extract; and an orally acceptable carrier; wherein Rebaudioside A has a purity of from about 95% to about 99%; and wherein the enzymatically Glucosylated Steviol Glycosides extract has a content in enzymatically Glucosylated Steviol Glycosides from 80% to 98% by weight, relative to the dry weight of the extract.

In another embodiment, there is provided an oral care composition, wherein the ratio of Rebaudioside A to enzymatically Glucosylated Steviol Glycosides extract is 1:1, or 1:2, or 1:3, or 1:4, or 1:5, or 2:1, or 3:1, or 4:1, or 5:1, respectively.

In another embodiment, there is provided an oral care composition, which is free from artificial sweeteners, wherein the artificial sweeteners comprise: saccharin, sucralose, aspartame, acesulfame K, advantame, sodium cyclamate, neotame and/or combinations thereof.

In another embodiment, there is provided an oral care composition, wherein the natural sweetener system is present in about 0.05% by weight to about 3% by weight, relative to the total weight of the oral care composition.

In another embodiment, there is provided an oral care composition, wherein the natural sweetener system comprises Rebaudioside A 98% pure and enzymatically Glucosylated Steviol Glycosides extract with a content in enzymatically Glucosylated Steviol Glycosides of 80% by weight, relative to the dry weight of the extract, in a (wt %) ratio of 1:2.

In another embodiment, there is provided an oral care composition, wherein the natural sweetener system comprises Rebaudioside A 98% pure and enzymatically Glucosylated Steviol Glycosides extract with a content in enzymatically Glucosylated Steviol Glycosides of 80% by weight, relative to the dry weight of the extract, in a (wt %) ratio of 4:1.

In another embodiment, there is provided an oral care composition, wherein the natural sweetener system comprises Rebaudioside A 98% pure and enzymatically Glucosylated Steviol Glycosides extract with a content in enzymatically Glucosylated Steviol Glycosides of 80% by weight, relative to the dry weight of the extract, in a (wt %) ratio of 5:1.

In one aspect, the enzymatically glucosylated steviol glycoside extract is available as an extract comprising 80% by weight to 95% by weight of enzymatically glucosylated steviol glycoside relative to the dry wt. % of the extract. For example, the remaining amount of the extract comprises steviosides and rebaudioside which does not react with the enzyme.

In yet another aspect, the natural sweetener system described herein may be used as sole sweetener system in oral care compositions or may be used in conjunction with other natural sweetener systems to give a cleaner and more balanced sweetness profile.

In one aspect, the disclosure provides an oral care composition comprising a natural sweetener system, wherein the natural sweetener system comprises a rebaudioside A extract with a purity of from 95% to 99% by wt., wherein the amount of rebaudioside A is relative to the dry weight of the rebaudioside A (Reb A) extract.

In another embodiment, the disclosure provides an oral care composition comprising a Reb A extract in a purity from 95% to 99% by wt., wherein the amount of Reb A is relative to the dry weight of the Reb A extract, and an enzymatically glucosylated steviol glycoside extract.

In another embodiment, the disclosure provides an oral care composition comprising a natural sweetener system comprising a Reb A extract in a purity of from 95% to 99% by wt., relative to the Reb A extract, and enzymatically glucosylated steviol glycosides extract, wherein the enzymatically glucosylated steviol glycosides extract are in 80% weight to 98% weight, relative to the dry wt. % of the enzymatically glucosylated steviol glycoside extract.

In another embodiment, the disclosure provides an oral care composition comprising a natural sweetener system comprising a Reb A extract in a purity of from 95% to 99%, relative to the Reb A extract, and enzymatically glucosylated steviol glycosides extract, wherein the enzymatically glucosylated steviol glycoside extract are in 80% weight to 98% by weight, wherein the weight is relative to the dry wt % of the enzymatically glucosylated steviol glycosides extract, and an orally acceptable carrier.

In some embodiments, the oral care composition of the disclosure, e.g., any of Composition 1.0 et seq., is in the form selected from: a toothpaste, a dentifrice, a mouthwash, a mouth rinse, mouth spray, a topical oral gel and a denture cleanser. In another embodiment, the oral care composition of the disclosure is in the form of dental strips, beads, varnish, tablets, toothpaste tape, toothpaste film, toothpaste ribbon, dental floss, chewing gum, gummies, lozenges or toothpowder.

In still further embodiments, the oral care compositions disclosed herein further comprise abrasives, stain prevention compounds, whitening agents, pigments, erosion prevention agents, opacifiers, antitartar compounds, polymer complexes, surfactants, humectants and/or combinations thereof. Optionally, the oral care compositions of the invention further comprise diluents, bicarbonate salts, pH modifying agents, foam modulators, thickening agents, flavoring agents, antibacterial agents, desensitizing agents, anticariogenic agents, fluoride ion sources, anticalculus agents, and/or combinations thereof. Optionally, the oral care composition comprises water. Optionally, the oral care compositions of the invention comprise cleaning agents, flavoring agents, sweetening agents, adhesion agents, surfactants, foam modulators, pH modifying agents, humectants, moisturizers, mouth feel agents, colorants, abrasives, preservatives, fluoride ion source, saliva stimulating agents, emollients, viscosity modifiers, diluents, emulsifiers, nutrients and/or combinations thereof.

In another embodiment, the disclosure provides an oral care composition comprising a natural sweetener system, wherein the natural sweetener system comprises a rebaudioside A extract and an enzymatically glucosylated steviol glycoside extract, wherein the rebaudioside A extract has a purity of from 95% to 99% by wt., relative to the dry weight of the extract, and the enzymatically glucosylated steviol glycosides extract has a content in enzymatically glucosylated steviol glycosides extract from 80% by weight to 98% by weight relative to the dry weight of the enzymatically glucosylated steviol glycosides extract. In a further embodiment, the disclosure provides an oral care composition comprising a natural sweetener system, wherein the sweetener system comprises a ratio (e.g., wt %) of rebaudioside A extract to enzymatically glucosylated steviol glycosides extract of: 1:1, or 1:2, or 1:3, or 1:4, or 1:5, or 2:1, or 3:1, or 4:1, or 5:1, respectively.

In one embodiment, the disclosure provides an oral care composition comprising from 0.05 wt. % to 3 wt. % of a natural sweetener system, where the wt % is relative to the total weight of the composition, and wherein the natural sweetener system comprises a rebaudioside An extract and an enzymatically glucosylated steviol glycoside extract, wherein the rebaudioside An extract has a purity of from 95% to 99% by wt., relative to the dry weight of the extract and the enzymatically glucosylated steviol glycosides extract has a content in enzymatically glucosylated steviol glycosides extract from 80% by weight to 98% by weight relative to the dry weight of the enzymatically glucosylated steviol glycosides extract.

In another embodiment, the disclosure provides an oral care composition, e.g., any of Composition 1.0 et seq., comprising a natural sweetener system, wherein the natural sweetener system comprises rebaudioside A extract and enzymatically glucosylated steviol glycosides extract, and wherein the weight ratio of the rebaudioside A extract to enzymatically glucosylated steviol glycosides extract is from: 1:1 to 1:6, e.g., about 1:1, or about 1:2, or about 1:3, or about 1:4, or about 1:5. In another the embodiment, the weight ratio of the rebaudioside A extract to enzymatically glucosylated steviol glycosides extract is from: 6:1 to 1:6, e.g., about 2:1, or about 3:1, or about 4:1, or about 5:1, respectively.

In another embodiment, the disclosure provides an oral care composition, e.g., any of Composition 1.0 et seq., comprising a natural sweetener system, wherein the natural sweetener system comprises rebaudioside A extract and enzymatically glucosylated steviol glycosides extract, wherein the composition is free from any artificial sweeteners, e.g., saccharin, sucralose, aspartame, acesulfame K, advantame, sodium cyclamate, neotame and/or combinations thereof.

In another embodiment, the disclosure provides an oral care composition, e.g., any of Composition 1.0 et seq., comprising a natural sweetener system, wherein the natural sweetener system comprises rebaudioside A extract with a purity from 95% to 99% by wt., relative to the weight of the rebaudioside A extract and the enzymatically glucosylated steviol glycosides extract has a content in enzymatically glucosylated steviol glycosides extract from 80% by weight to 98% by weight relative to the dry weight of the enzymatically glucosylated steviol glycosides extract; and wherein the natural sweetener system is present from 0.05% by weight to 3% by weight, relative to the total weight of the oral care composition.

In another embodiment, the disclosure provides an oral care composition, e.g., any of Composition 1.0 et seq., comprising a natural sweetener system, wherein the natural sweetener system comprises a Rebaudioside A 98% by wt. pure extract, relative to the weight of the Rebaudioside A extract, and enzymatically glucosylated steviol glycosides extract with a content in enzymatically glucosylated steviol glycosides extract of 80% by weight, relative to the weight of the extract, in a weight ratio of about 1:2 (Rebaudioside A extract: enzymatically glucosylated steviol glycosides extract).

In another embodiment, the disclosure provides an oral care composition, e.g., any of Composition 1.0 et seq., comprising a natural sweetener system, wherein the natural sweetener system comprises Rebaudioside A 98% pure extract or greater (e.g., about 98% pure), relative to the weight of the Rebaudioside A extract, and enzymatically glucosylated steviol glycosides extract with a content in enzymatically glucosylated steviol glycosides extract of 80% by weight, relative to the weight of the extract, in a ratio (wt %) of about 4:1.

In another embodiment, the disclosure provides an oral care composition, e.g., any of Composition 1.0 et seq., comprising a natural sweetener system, wherein the natural sweetener system comprises a Reb A 98% by wt. pure extract or greater (e.g., about 98% pure), relative to the weight of the Rebaudioside A extract, and enzymatically glucosylated steviol glycosides extract with a content in enzymatically glucosylated steviol glycosides extract of 80% by weight, relative to the weight of the extract, in a ratio (wt %) of about 5:1.

In another embodiment, the disclosure provides an oral care composition, e.g., any of Composition 1.0 et seq., comprising a natural sweetener system, wherein the natural sweetener system comprises Rebaudioside A extract and an enzymatically glucosylated steviol glycosides extract, and wherein the composition further comprises an orally acceptable carrier.

In another embodiment, the disclosure provides an oral care composition, e.g., any of Composition 1.0 et seq., comprising a natural sweetener system, wherein the natural sweetener system comprises Rebaudioside A extract and an enzymatically glucosylated steviol glycosides extract, and wherein the composition further comprises an ingredient selected from: abrasives, stain prevention compounds, antitartar compounds, whitening agents, anticalculus compounds, polymer complexes, surfactants, humectants and combinations thereof.

In another embodiment, the disclosure provides an oral care composition, e.g., any of Composition 1.0 et seq., comprising a natural sweetener system, wherein the natural sweetener system comprises Rebaudioside A extract and an enzymatically glucosylated steviol glycosides extract, wherein the composition further comprises an orally acceptable carrier selected from: pH modifying agents, foam modulators, thickening agents, flavoring agents, antibacterial agents, desensitizing agents, anticariogenic agents, fluoride ion sources, anticalculus agents, and combinations thereof.

In another embodiment, the disclosure provides an oral care composition, e.g., any of Composition 1.0 et seq., comprising a natural sweetener system, wherein the natural sweetener system comprises Rebaudioside A extract and an enzymatically glucosylated steviol glycosides extract, and wherein the composition further comprises an ingredient selected from: nutrients, vitamins, antibacterial agents and combinations thereof.

In another embodiment, the disclosure provides an oral care composition, e.g., any of Composition 1.0 et seq., comprising a natural sweetener system, wherein the natural sweetener system comprises Rebaudioside A extract and an enzymatically glucosylated steviol glycosides extract, and wherein the composition comprises an abrasive selected from: silica gel, hydrated silica, high cleaning silica, core shell silica, precipitated silica, calcium carbonate, calcium pyrophosphate and combinations thereof.

In another embodiment, the disclosure provides an oral care composition, e.g., any of Composition 1.0 et seq., comprising a natural sweetener system, wherein the natural sweetener system comprises Rebaudioside A extract and an enzymatically glucosylated steviol glycosides extract, and wherein the oral care composition is in the form selected from: dentifrice (e.g., a toothpaste), mouthwash, mouth rinse, mouth spray, topical oral gel and denture cleanser.

In another embodiment, the disclosure provides an oral care composition, e.g., any of Composition 1.0 et seq., comprising a natural sweetener system, wherein the natural sweetener system comprises a Rebaudioside A extract and an enzymatically glucosylated steviol glycosides extract, and wherein the oral care composition is in the form selected from: dental strips, beads, varnish, toothpaste tape, toothpaste film, toothpaste ribbon, dental floss, tablets, chewing gum, gummies, lozenges and toothpowder.

In another embodiment, the disclosure provides an oral care composition comprising a natural sweetener system, wherein the natural sweetener system comprises Rebaudioside A extract and an enzymatically glucosylated steviol glycosides extract, wherein the composition further comprises a fluoride ion source.

In another embodiment, the disclosure provides an oral care composition, e.g., any of Composition 1.0 et seq., comprising a natural sweetener system, wherein the natural sweetener system comprises a Rebaudioside A extract and an enzymatically glucosylated steviol glycosides extract, and wherein the oral care composition further comprises a zinc ion source (e.g., zinc citrate, or zinc oxide, or zinc phosphate and combinations thereof).

In another embodiment, the disclosure provides an oral care composition, e.g., any of Composition 1.0 et seq., comprising a natural sweetener system, wherein the natural sweetener system comprises a Rebaudioside A extract and an enzymatically glucosylated steviol glycosides extract, and wherein the oral care composition further comprises a stannous ion source (e.g., stannous fluoride, or stannous chloride, or stannous pyrophosphate and combinations thereof).

In another embodiment, the disclosure provides an oral care composition, e.g., any of Composition 1.0 et seq., comprising a natural sweetener system, wherein the natural sweetener system comprises a Rebaudioside A extract and an enzymatically glucosylated steviol glycosides extract, and wherein the oral care composition further comprises xylitol.

In another embodiment, the disclosure provides an oral care composition, e.g., any of Composition 1.0 et seq., comprising a natural sweetener system, wherein the natural sweetener system comprises a Rebaudioside A extract and an enzymatically glucosylated steviol glycosides extract; and wherein the Rebaudioside A extract has a purity of from 95% to 99% by wt. relative to the dry weight of the Rebaudioside A extract; and the enzymatically glucosylated steviol glycosides extract has a content in enzymatically glucosylated steviol glycosides extract from 80% by weight to 98% by weight relative to the dry weight of the enzymatically glucosylated steviol glycosides extract.

In another embodiment, the disclosure provides an oral care composition, e.g., any of Composition 1.0 et seq., comprising a natural sweetener system, wherein the natural sweetener system comprises a Rebaudioside A extract and an enzymatically glucosylated steviol glycosides extract, and wherein the ratio (wt %) of Rebaudioside A to enzymatically glucosylated steviol glycosides extract is 1:2, or 4:1, or 5:1, respectively.

In one aspect, the disclosure provides an oral care composition (Composition 1.0) comprising a natural sweetener system, wherein the natural sweetener system comprises:
An enzymatically glucosylated steviol glycoside (“GSG”) extract (e.g., an extract comprising 80% by weight to 95% by weight of enzymatically glucosylated steviol glycoside) (e.g., about 80% pure GSG); and
A rebaudioside A (“Reb A”) extract (e.g., where the Reb A extract has a purity from 95% to 99% by wt., wherein the wt. % of rebaudioside A is relative to the dry weight of the rebaudioside A (Reb A) extract) (e.g., about 98% pure Reb A).

For example, Composition 1.0 also includes the following:
1.1 The oral care composition of 1.0, wherein the weight ratio of Rebaudioside A extract to enzymatically glucosylated steviol glycosides extract is from 1:1 to 1:6, e.g., about 1:1, or about 1:2, or about 1:3, or about 1:4, or about 1:5; alternatively about 2:1, or about 3:1, or about 4:1, or 5:1, respectively.
1.2 The oral care composition of Composition 1.0 or 1.1, wherein the natural sweetener system is present in about 0.05% by weight to about 3% by weight, relative to the total weight of the oral care composition.
1.3 The oral care composition of any of the preceding compositions, wherein the Reb A extract is present in an amount from 0.1-1% by wt., relative to the wt % of the natural sweetener system (e.g., about 0.35% by wt.).
1.4 The oral care composition of any of the preceding compositions, wherein the Reb A extract is present in an amount from 0.1-1% by wt., relative to the weight of the natural sweetener system (e.g., about 0.2% by wt.). (e.g., about 0.35% by wt.) (e.g., about 0.4% by wt.).
1.5 The oral care composition of any of the preceding compositions, wherein the GSG extract is present in an amount from 0.1-1% by wt., relative to the weight of the natural sweetener system (e.g., about 0.5% by wt.) (e.g., about 0.6% by wt.) (e.g., about 0.7% by wt.) (e.g., about 0.75% by wt.) (e.g., about 0.8% by wt).
1.6 The oral care composition of any of the preceding compositions, wherein the Reb A extract (e.g., about 98% pure Reb A) is present in an amount from 0.1-1% by wt. of the natural sweetener system (e.g., about 0.2% by wt.). (e.g., about 0.35% by wt.) (e.g., about 0.4% by wt.); and wherein the GSG extract (e.g., GSG80) is present in an amount from 0.1-1% by wt. of the natural sweetener system (e.g., about 0.5% by wt.) (e.g., about 0.6% by wt.) (e.g., about 0.7% by wt.) (e.g., about 0.75% by wt).
1.7 The oral care composition according to any of the preceding compositions, wherein the natural sweetener system comprises Reb A 95%-98% by wt. pure extract (e.g., 98% pure or greater), wherein the wt % of Reb A is relative to the total weight of the Reb A extract (e.g., 98% pure Reb A), and enzymatically glucosylated steviol glycosides (“GSG”) extract of 80%-95% by weight, relative to the weight of the GSG extract (e.g., GSG80), and wherein the weight ratio of Reb A extract to GSG extract (e.g., the wt % of each relative to the total wt of the composition) from 1:1 to 1:6, e.g., about 1:1, or about 1:2, or about 1:3, or about 1:4, or about 1:5; alternatively about 2:1, or about 3:1, or about 4:1, or about 5:1, respectively (e.g., 1:2).
1.8 Any of the preceding oral care compositions, wherein the oral care composition comprises a stannous ion source.
1.9 Any of the preceding oral care compositions, wherein the stannous ion source is selected from the group consisting of: stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and combinations thereof.
1.10 Any of the preceding compositions, wherein the stannous ion source comprises stannous fluoride.
1.11 Any of the preceding compositions, wherein the stannous ion source comprises stannous fluoride in an amount of 0.1 wt. % to 2 wt. % (0.1 wt %-0.6 wt. %) (e.g., about 0.454 wt. %) of the total composition weight.
1.12 Any of the preceding compositions, wherein the stannous ion source comprises stannous fluoride in an amount from 50 to 25,000 ppm (e.g., 750-7000 ppm, e.g., 1000-5000 ppm, e.g., about 4500 ppm, e.g., about 4540 ppm).
1.13 Any of the preceding compositions, wherein the composition comprises stannous fluoride and stannous pyrophosphate.
1.14 Any of the preceding compositions, wherein the composition comprises stannous fluoride and stannous chloride.
1.15 Any of the preceding compositions, wherein the one or more stannous ion source(s) is in an amount from 0.1%-5% by wt. of the total composition.
1.16 Any of the preceding compositions, wherein the composition comprises a zinc ion source and wherein the zinc ion source comprises one or more zinc salt(s) selected from the group consisting of: zinc citrate, zinc oxide, zinc phosphate, zinc lactate, zinc sulfate, zinc silicate, zinc gluconate and combinations thereof.
1.17 Any of the preceding compositions, wherein the composition comprises a zinc ion source and wherein the zinc ion source comprises zinc oxide.
1.18 Any of the preceding compositions, wherein the composition comprises a zinc ion source and wherein the zinc ion source comprises zinc citrate.
1.19 Any of the preceding compositions, wherein the composition comprises a zinc ion source and wherein the zinc ion source comprises zinc oxide and zinc citrate.
1.20 Any of the preceding compositions, wherein the ratio of the amount of zinc oxide (e.g., wt. %) to zinc citrate (e.g., wt %) is from 1.5:1 to 4.5:1 (e.g., 2:1, 2.5:1, 3:1, 3.5:1, or 4:1).
1.21 Any of the preceding compositions, wherein the zinc citrate is in an amount of from 0.25 to 0.75 wt % (e.g., 0.5 wt. %) and zinc oxide may be present in an amount of from 0.75 to 1.25 wt % (e.g., 1.0 wt. %) based on the weight of the oral care composition.
1.22 Any of the preceding compositions wherein the zinc citrate is about 0.5 wt %.
1.23 Any of the preceding compositions wherein the zinc oxide is about 1.0 wt %.
1.24 Any of the preceding compositions where the zinc citrate is about 0.5 wt % and the zinc oxide is about 1.0 wt %.
1.25 Any of the preceding compositions, wherein the composition comprises a zinc ion source and wherein the zinc ion source comprises zinc phosphate (e.g., wherein the zinc phosphate is a preformed salt of zinc phosphate) (e.g., zinc phosphate hydrate) (e.g., from 0.5-4 wt % of zinc phosphate) (e.g., about 1.0 wt % of zinc phosphate).
1.26 The preceding composition, wherein the zinc phosphate is added as a preformed salt.
1.27 Any of the preceding compositions, wherein the composition comprises a source of zinc ions, and wherein the zinc ion source comprises zinc lactate.
1.28 Any of the preceding compositions, wherein the zinc ion source is in an amount from 0.1%-5% by wt. of the total composition (e.g., zinc phosphate from 0.1%-5% by wt. of the total composition).
1.29 Any preceding composition comprising an effective amount of a fluoride ion source.
1.30 The preceding composition, wherein the amount of the fluoride ion source is in an amount from 0.01% to 5% by weight, relative to the weight of the oral care composition, for example, from 0.05 to 4% by weight, or from 0.1% to 3% by weight, or from 0.2 to 2% by weight, or from 0.3 to 1% by weight, or from 0.3 to 0.5% by weight, or about 0.32% by weight (e.g., 0.32% by weight).
1.31 Any of the preceding compositions, wherein the fluoride source is selected from the group consisting of: sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides and combinations thereof.
1.32 The preceding composition, wherein the fluoride ion source comprises sodium fluoride (e.g., from 0.2%-2% by wt. of sodium fluoride)
1.33 The composition of 1.31, wherein the fluoride ion source comprises stannous fluoride (e.g., stannous fluoride from 0.1%-2% by wt. of the total composition).
1.34 The composition of 1.31, wherein the fluoride ion source comprises sodium monofluorophosphate.
1.35 Any preceding composition, wherein the composition comprises water in the amount of 10% by weight or more, relative to the weight of the oral care composition, for example, 10-90%, or 10-80%, or 10-70%, or 10-60%, or 10-50%, or 10-40%, or or 15-30%, 15%-40% 20%-40%, 20-35%, or 20-50%, or 30-35%, or about 25% or about 30%, by weight of the composition.
1.36 Any preceding composition, further comprising an organic buffer system, wherein the buffer system comprises a carboxylic acid and one or more conjugate base salts thereof, for example, alkali metal salts thereof (e.g., citric acid and sodium citrate).
1.37 Any preceding composition, wherein the composition comprises the organic acid buffer system in an amount of 0.1 to 5.0% by weight of the composition, measured as the combined amount of organic acid and any conjugate base salts (e.g., citric acid and sodium citrate); for example, from 0.5 to 4.0%, or from 1.0 to 3.0%, or from 1.5 to 3.0%, or from 1.0 to 2.4%, or from 1.0% to 2.0%, or from 1.0% to 1.5%, or about 1.2%, by weight of the composition.
1.38 Any preceding composition, wherein the oral care composition further comprises an abrasive, for example, silica abrasives, calcium abrasives, and other abrasives as disclosed herein.
1.39 Any preceding composition, further comprising one or more humectants, as described herein, e.g., selected from sorbitol, glycerol, xylitol and propylene glycol, or combinations thereof, e.g., a combination of sorbitol and glycerin.
1.40 Any of the preceding compositions, wherein the zwitterionic surfactant comprises cocamidopropyl betaine, (e.g., in an amount of 0.1-5% by weight) (e.g., about 0.6% by wt.).
1.41 Any preceding composition, further comprising an effective amount of one or more alkali phosphate salts for example orthophosphates, pyrophosphates, tripolyphosphates, tetraphosphates or higher polyphosphates.
1.42 The preceding composition, wherein the alkali phosphate salts comprise tetrasodium pyrophosphate or tetrapotassium pyrophosphate, for example, in an amount of 0.5 to 5% by weight of the composition, e.g., 1-4%, or about 2-4%, or about 1-2% or about 1.5% or about 2% or about 4%, by weight.
1.43 The preceding composition, wherein the alkali phosphate salts comprise sodium tripolyphosphate or potassium tripolyphosphate, for example, in an amount of 0.5 to 6% by weight of the composition, e.g., 1-4%, or 2-3% or about 3% by weight.
1.44 Any preceding composition, further comprising a whitening agent.
1.45 Any preceding composition, wherein the oral care composition is in the form selected from: dentifrice (e.g., a toothpaste or oral gel), powder (e.g., tooth powder), cream, mouthwash, strip or gum (e.g., chewing gum).
1.46 Any preceding composition, wherein the pH of the composition is from 6 to 9, such as from 6.5 to 8, or from 6.5 to 7.5, or about 7.0.
1.47 Any preceding composition, wherein the composition is a single-phase composition (e.g., not a dual-phase composition).
1.48 Any preceding composition, wherein the composition is essentially free or free of phosphates of more than four phosphate groups.
1.49 Any preceding composition, wherein the composition is essentially free or free of phosphates of more than three phosphate groups.
1.50 Any preceding composition, wherein the composition is essentially free or free of hexametaphosphate salts (e.g., sodium hexametaphosphate).
1.51 Any of the preceding compositions, wherein the composition is effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health, e.g., by reducing potential for systemic infection via the oral tissues.
1.52 Any preceding compositions, wherein the composition further comprises a polymer selected from the group consisting of: carboxymethyl cellulose (free form or a salt, e.g., sodium salt), a gum (e.g., xanthan gum, carrageenan gum, or gum arabic), polyethylene glycol (e.g., polyethylene glycol 200, 400, 600 or 800, or a mixture thereof), and combinations thereof, for example, a mixture of sodium carboxy methyl cellulose, xanthan gum, polyethylene glycol 600.
1.53 Composition 1.52, wherein the polymer comprises sodium carboxy methyl cellulose.
1.54 Composition 1.52, wherein the polymer comprises xanthan gum.
1.55 Any preceding composition further comprising a silica thickener and/or a silica abrasive.
1.56 Any preceding composition, wherein the oral care composition comprises an additional anionic surfactant that is not sodium lauryl sulfate, wherein the additional anionic surfactant is selected from the group consisting of: water-soluble salts of higher fatty acid monoglyceride monosulfates (such as the sodium salt of the monosulfated monoglyceride of hydrogenated coconut oil fatty acids such as sodium N-methyl N-cocoyl taurate), sodium cocomonoglyceride sulfate, higher alkyl-ether sulfates (e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na)), higher alkyl aryl sulfonates such as sodium dodecyl benzene sulfonate (sodium lauryl benzene sulfonate), higher alkyl sulfoacetates (such as sodium lauryl sulfoacetate (dodecyl sodium sulfoacetate)), higher fatty acid esters of 1,2 dihydroxy propane sulfonate, sulfocolaurate (N-2-ethyl laurate potassium sulfoacetamide) and sodium lauryl sarcosinate.
1.57 Any of the preceding compositions wherein the composition comprises an amino acid.
1.58 The preceding composition wherein the amino acid is a basic amino acid (e.g., arginine)
1.59 Any of the preceding compositions wherein the amino acid is a basic amino acid provided in the form of a di- or tri-peptide comprising arginine or lysine, or salts thereof.
1.60 Any of the preceding compositions wherein the basic amino acid comprises arginine or lysine, and wherein the arginine or lysine is present in an amount corresponding to 1% to 15%, e.g., 3 wt. % to 10 wt. % of the total composition weight, about e.g., 1.5%, 4%, 5%, or 8%, wherein the weight of the basic amino acid is calculated as free form.
1.61 Any of the preceding compositions wherein the amino acid comprises arginine from 0.1 wt. %-6.0 wt. % (e.g., about 1.5 wt %) (e.g., about 5 wt %) of the total composition, wherein the weight of the arginine is calculated as free form.
1.62 Any of the preceding compositions wherein the amino acid is arginine from about 1.5 wt. of the total composition, wherein the weight of the arginine is calculated as free form.
1.63 Any of the preceding compositions wherein the amino acid is arginine from 4.5 wt. %-8.5 wt. % (e.g., about 5.0 wt. %) of the total composition, wherein the weight of the basic amino acid is calculated as free form.
1.64 Any of the preceding compositions wherein the amino acid is arginine from about 5.0 wt. % of the total composition, wherein the weight of the basic amino acid is calculated as free form.
1.65 Any of the preceding compositions wherein the amino acid is arginine from 3.5 wt. %-9 wt. % of the total composition, wherein the weight of the basic amino acid is calculated as free form.
1.66 Any of the preceding compositions wherein the amino acid is L-arginine.
1.67 Any of the preceding compositions wherein the amino acid is a free form arginine.
1.68 Any of the preceding compositions wherein the amino acid is arginine or lysine in partially or wholly in salt form.
1.69 Composition 1.68 wherein the amino acid is arginine phosphate.
1.70 Composition 1.68 wherein the amino acid is arginine hydrochloride.
1.71 Composition 1.68 wherein the amino acid is arginine bicarbonate.
1.72 Any of the preceding compositions wherein the amino acid is arginine or lysine ionized by neutralization with an acid or a salt of an acid.
1.73 Any foregoing composition comprising a zwitterionic surfactant.
1.74 The preceding composition, wherein the zwitterionic surfactant is a betaine zwitterionic surfactant (e.g., from 0.1%-5% by wt. of the total composition) (e.g., 0.2%-1% by wt. of the total composition) (e.g., about 0.6% by wt. of the total composition).
1.75 The preceding composition, wherein the betaine zwitterionic surfactant is a C8-C16 aminopropyl betaine (e.g., cocamidopropyl betaine).
1.76 The preceding composition wherein the C8-C16 aminopropyl betaine is cocamidopropyl betaine.
1.77 The preceding composition wherein the cocamidopropyl betaine, is present in an amount of from 0.5% to 4% by wt of the total composition.
1.78 The preceding composition, wherein the cocamidopropyl betaine is from 0.1% to 3% by wt of the total composition.
1.79 The preceding composition wherein the cocamidopropyl betaine is from 0.1% to 1% (e.g., about 0.6% by wt. of the total composition).
1.80 Any of the preceding compositions wherein the composition comprises cocamidopropyl betaine and sodium methyl cocoyl taurate in a wt % ratio of (e.g., wt %) is from 0.1:1 to 1:1 (e.g., 0.1:1, 0.2:1, 0.3:1, 0.4:1 or 0.5:1) (e.g., 0.3:1)
1.81 Any preceding composition, wherein the oral care composition is free of sodium lauryl sulfate.
1.82 Any preceding composition, wherein the composition comprises:
   Zinc phosphate (e.g., from 0.5%-4% by wt.);
   Stannous fluoride;
   A natural sweetener system comprising:
   An enzymatically glucosylated steviol glycoside (“GSG”) extract wherein the extract comprises 80% to 95% by weight of an enzymatically glucosylated steviol glycoside, wherein the wt. % of GSG is relative to the dry wt % of the extract; and
   A rebaudioside A (“Reb A”) extract (e.g., where the Reb A extract has a purity from 95% to 99% by wt. %, wherein the wt. % of rebaudioside A is relative to the dry weight of the rebaudioside A (Reb A) extract) (e.g., about 98% pure Reb A); and
   An orally acceptable carrier.
1.83 Any preceding composition, wherein the composition comprises:
   Zinc phosphate (e.g., from 0.5%-4% by wt.);
   Stannous fluoride (e.g., from 0.2%-2% by wt.);
   Stannous pyrophosphate (e.g., from 0.1-2% by wt.);
   A natural sweetener system comprising:
      An enzymatically glucosylated steviol glycoside (“GSG”) extract wherein the extract comprises 80% to 95% by weight of an enzymatically glucosylated steviol glycoside, wherein the wt. % of GSG is relative to the dry wt % of the extract; and
      A rebaudioside A (“Reb A”) extract (e.g., where the Reb A extract has a purity from 95% to 99% by wt., wherein the wt. % of rebaudioside A is relative to the dry weight of the rebaudioside A (Reb A) extract) (e.g., about 98% pure Reb A); and
   An orally acceptable carrier.
1.84 Any preceding composition, wherein the composition comprises:
   Zinc phosphate (e.g., from 0.5%-4% by wt.);
   Stannous fluoride (e.g., from 0.2%-2% by wt.);
   Arginine (e.g., from 0.5%-5 wt. %);
   A natural sweetener system comprising:
      An enzymatically glucosylated steviol glycoside (“GSG”) extract wherein the extract comprises 80% to 95% by weight of an enzymatically glucosylated steviol glycoside, wherein the wt. % of GSG is relative to the dry wt % of the extract; and
      A rebaudioside A (“Reb A”) extract (e.g., where the Reb A extract has a purity from 95% to 99% by wt., wherein the wt. % of rebaudioside A is relative to the dry weight of the rebaudioside A (Reb A) extract) ((e.g., about 98% pure Reb A); and
   An orally acceptable carrier.
1.85 Any preceding composition, wherein the composition comprises:
   Zinc citrate (e.g., from 0.25-0.75 by wt.);
   Zinc oxide (e.g., from 0.50-1.5 by wt.);
   A natural sweetener system comprising:
      An enzymatically glucosylated steviol glycoside (“GSG”) extract wherein the extract comprises 80% to 95% by weight of an enzymatically glucosylated steviol glycoside, wherein the wt. % of GSG is relative to the dry wt % of the extract; and
      A rebaudioside A (“Reb A”) extract (e.g., where the Reb A extract has a purity from 95% to 99% by wt., wherein the wt. % of rebaudioside A is relative to the dry weight of the rebaudioside A (Reb A) extract) (e.g., about 98% pure Reb A); and
   An orally acceptable carrier.
1.86 Any preceding composition, wherein the composition comprises:
   Zinc citrate (e.g., from 0.25%-0.75% by wt.);
   Zinc oxide (e.g., from 0.5%-1.5% by wt.);
   Arginine (e.g., from 0.5%-5 wt. %);
   A natural sweetener system comprising:
      An enzymatically glucosylated steviol glycoside (“GSG”) extract wherein the extract comprises 80% to 95% by weight of an enzymatically glucosylated steviol glycoside, wherein the wt. % of GSG is relative to the total wt % of the extract; and
      A rebaudioside A (“Reb A”) extract (e.g., where the Reb A extract has a purity from 95% to 99% by wt., wherein the wt. % of rebaudioside A is relative to the total weight of the rebaudioside A (Reb A) extract (e.g., about 98% pure Reb A); and
   An orally acceptable carrier.
1.87 Any of the compositions of 1.81-1.86 further comprising cocamidopropyl betaine, in an amount of from 0.1% to 5% by wt of the total composition.
1.88 Any preceding composition wherein the composition does not contain any sodium lauryl sulfate.
1.89 Any of composition 1.0-1.87 wherein the composition is substantially free of sodium lauryl sulfate.
1.90 Any of the preceding compositions, wherein the oral care composition is a dentifrice (e.g., a toothpaste or oral gel), powder (e.g., tooth powder), cream, mouthwash, strip or gum (e.g., chewing gum).
1.91 Any of the preceding compositions further comprising a preservative selected from: benzyl alcohol, Methylisothizolinone (“MIT”), Sodium bicarbonate, lauryl alcohol, and polyphosphate.
1.92 Any of the preceding compositions comprising nitric acid or a water-soluble nitrate salt (e.g., potassium nitrate).
1.93 The preceding composition, wherein the water-soluble nitrate salt is selected from an alkali or alkaline earth metal nitrate, or zinc nitrate, silver nitrate, or ammonium nitrate.
1.94 The preceding composition, wherein the water-soluble nitrate salt is an alkali metal nitrate salt or an alkaline earth metal nitrate salt.
1.95 The preceding composition, wherein the nitrate salt is selected from lithium nitrate, sodium nitrate, potassium nitrate, magnesium nitrate, and calcium nitrate.
1.96 The preceding composition, wherein the nitrate salt is potassium nitrate.
1.97 Any preceding composition, wherein the oral care composition is free or substantially free of sodium lauryl sulfate.
1.98 Any of the preceding compositions comprising an effective amount of a taurate surfactant, wherein the taurate surfactant is represented by Formula (1):

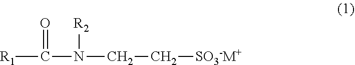

wherein R₁ is a saturated or unsaturated, straight or branched alkyl chain with 6 to 18 C atoms R₂ is H or methyl, and M⁺ is H, sodium, or potassium (e.g., sodium methyl cocoyl taurate).
1.99 Composition 1.98, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations thereof.
1.100 Any of the preceding compositions, wherein the taurate surfactant comprises one or more surfactant selected from the group consisting of: sodium lauroyl methyl taurate (or sodium methyl lauroyl taurate), sodium methyl cocoyl taurate (SMCT), and combinations thereof.
1.101 Any of the preceding compositions, wherein the taurate surfactant comprises sodium methyl cocoyl taurate (e.g., 1%-5% by wt of sodium methyl cocoyl taurate) (e.g., about 2% by wt. sodium methyl cocoyl taurate).
1.102 Any of the preceding compositions, wherein the taurate surfactant is present in an amount of from 0.25% to 5%, e.g., from 0.4% to 3%, e.g., from 0.4% to 2.75%, e.g., from 0.4% to 2.5%, e.g., from 0.5% to 3%, e.g., from 0.8% to 3%, e.g., from 1% to 3%, e.g., from 1.2% to 2.7%, e.g., from 1.5% to 3%, e.g., from 2% to 3%, e.g., from 1% to 2.8%, e.g., from 1% to 2.7%, e.g., from 1% to 2.5%, e.g., from 1.5% to 2.8%, e.g., from 1.5% to 2.5%, e.g., from 1.8% to 3%, e.g., from 1.8% to 2.8%, e.g., from 1.8% to 2.7%, e.g., from 1.8% to 2.5%, e.g., about 2% by weight of the composition.
1.103 The oral care composition according to any of the preceding claims, wherein the oral care composition is free from artificial sweeteners, and wherein the artificial sweeteners comprise: saccharin, sucralose, aspartame, acesulfame K, advantame, sodium cyclamate, neotame and/or combinations thereof.
1.104 The oral care composition according to any of the preceding claims, wherein the weight ratio of the rebaudioside A extract to enzymatically glucosylated steviol glycosides extract is from: 6:1 to 1:1, e.g., about 2:1, or about 3:1, or about 4:1, or about 5:1, respectively.

In one aspect, the disclosure provides for oral care compositions, e.g., any of Composition 1.0 et seq., comprising a natural sweetener system. The natural sweetener system of the invention comprises Reb A and GSG extract. In one aspect, the purity of the Reb A is in a range of from 95% to 99%. The purity of Reb A in the oral care compositions described herein is about 95% pure, or about 96% pure, or about 97% pure, or about 98% pure, or about 99% pure. In one aspect the Reb A in the oral care compositions described herein, e.g., any of Composition 1.0 et seq, is 98% pure Reb A (e.g., about 98% pure Reb A).

In another aspect, the disclosure provides for oral care compositions, e.g., any of Composition 1.0 et seq., where the content in GSG within the extract is from 80% to 98% by weight, relative to the weight of the extract. In one aspect, the content in GSG in the extract is about 80% by weight, relative to the wt. of the extract, or the content in the GSG in the extract may be about 85% by weight, relative to the wt. of the extract, or the content in GSG in the extract may be about 90% by weight, relative to the wt. of the extract, or the content in GSG in the extract may be about 95% by weight, relative to the wt. of the extract, or the content in GSG in the extract may be about 98% by weight, relative to the wt. of the extract.

As used herein, an extract of GSG, wherein the content in enzymatically Glucosylated Steviol Glycosides is 80% by weight relative to the total weight of the GSG extract, will be referred to as GSG80. The extract of GSG, wherein the content in enzymatically Glucosylated Steviol Glycosides is 95% by weight (e.g., about 95% by wt.), relative to the wt. of the extract, will be referred to as GSG95. The number following “GSG” refers to the content in % by weight in the enzymatically Glucosylated Steviol Glycosides within the GSG extract.

In some embodiments, the disclosure provides for oral care compositions, e.g., any of Composition 1.0 et seq., where the natural sweetener system of the invention comprises a combination of Reb A 98% pure (e.g., about 98% pure) and GSG80.

In some embodiments, the disclosure provides for oral care compositions, e.g., any of Composition 1.0 et seq., where the oral care composition comprises a natural sweetener system comprising Rebaudioside A and an enzymatically Glucosylated Steviol Glycosides extract; an orally acceptable carrier; wherein Rebaudioside A has a purity of from 95% to 99% (e.g., from 97%-99% by wt.); and the enzymatically Glucosylated Steviol Glycosides extract has a content in enzymatically Glucosylated Steviol Glycosides from 80% by weight to 98% by weight, relative to the total weight of the extract.

In some embodiments, the disclosure provides for oral care compositions, e.g., any of Composition 1.0 et seq., wherein the natural sweetener system of the disclosure comprises a combination of Reb A 98% pure and GSG95. In some embodiments, the natural sweetener system has a ratio of Reb A to GSG95 is from 1:1 to 6:1, e.g., 1:1, or 1:2, or 1:3, or, 1:4, or, 1:5; alternatively about 2:1, or about 3:1, or about 4:1, or about 5:1, respectively. In some embodiments the natural sweetener system comprises Reb A 98% pure and GSG95 in a ratio of 1:2.

In some embodiments, the disclosure provides for oral care compositions, e.g., any of Composition 1.0 et seq., comprising a natural sweetener composition, wherein the natural sweetener system comprises a combination of xylitol, Reb A 98%, and GSG95.

In some embodiments, the disclosure provides for oral care compositions, e.g., any of Composition 1.0 et seq., comprising a natural sweetener composition, wherein the natural sweetener system of the disclosure comprises a combination of xylitol, Reb A 98%, and GSG95, wherein the ratio Reb A to GSG95 is 1:2.

In some embodiments, the disclosure provides for oral care compositions, e.g., any of Composition 1.0 et seq., comprising a natural sweetener composition, wherein the natural sweetener system is present from about 0.05% to about 3% weight in the oral care composition, wherein the % weight is based on the total weight of the oral care composition. In some embodiments, the disclosure provides for oral care compositions, e.g., any of Composition 1.0 et seq., where the natural sweetener system of the invention is present from about 0.05% to about 3% weight in the oral care composition, wherein the ratio (wt %) of Reb A to GSG is from 1:1 to 6:1, e.g., 1:1, or 1:2, or 1:3, or, 1:4, 1:5; alternatively the ratio (wt %) of Reb A to GSG is from 6:1 to 1:1, e.g., about 2:1, or about 3:1, or about 4:1, or about 5:1, respectively.

In some embodiments, the disclosure provides for oral care compositions, e.g., any of Composition 1.0 et seq., comprising a natural sweetener composition, wherein the natural sweetener system is present from about 0.05% to about 3% weight in the oral care composition, wherein the ratio (wt %) of Reb A to GSG80 is from 1:1 to 6:1, e.g., about 1:1, or about 1:2, or about 1:3, or, about 1:4, or, about 1:5. Alternatively, in some embodiments, e.g., any of Composition 1.0 et seq, the ratio (wt %) of Reb A to GSG is from 6:1 to 1:1, e.g., about 2:1, or about 3:1, or about 4:1, or about 5:1, respectively.

In some embodiments, the disclosure provides for oral care compositions, e.g., any of Composition 1.0 et seq., where the natural sweetener system is present from about 0.05% to about 3.00% weight in the oral care composition, wherein the ratio (wt %) of Reb A 98% to GSG95 is 1:1 to 6:1, e.g., 1:1, or 1:2, or 1:3, or, 1:4, or, 1:5, or 2:1, or 3:1, or 4:1, or 5:1, respectively.

In some embodiments, the disclosure provides for oral care compositions, e.g., any of Composition 1.0 et seq., where the natural sweetener system is present from about 0.05% to about 3.00% weight in the oral care composition, wherein the ratio (wt %) of Reb A 98% to GSG98 is 1:1 to 6:1, e.g., 1:1, or 1:2, or 1:3, or, 1:4, or, 1:5, or 2:1, or 3:1, or 4:1, or 5:1, respectively.

In some embodiments, the disclosure provides for oral care compositions, e.g., any of Composition 1.0 et seq., where oral care compositions comprise a natural sweetener system, as described herein in an amount of about 0.1%, or about 0.2%, or about 0.3%, or about 0.4%, or about 0.5%, or about 0.6%, or about 0.7%, or about 0.8%, or about 0.9%, or about 1%, or about 1.1%, or about 1.2%, or about 1.3%, or about 1.4%, or about 1.5%, or about 1.6%, or about 1.7%, or about 1.8%, or about 1.9%, or about 2%, or about 2.1%, or about 2.2%, or about 2.3%, or about 2.4%, or about 2.5%, or about 2.6%, or about 2.7%, or about 2.8%, or about 2.9% or about 3% by weight relative to the weight of the oral care compositions thereof.

In some embodiments, the disclosure provides for oral care compositions, e.g., any of Composition 1.0 et seq., where the natural sweetener system comprises a combination of about 0.05% to about 10% weight xylitol, and about 0.05% to about 3% weight natural sweetener system of the invention, wherein the % weight are based on the total weight of the oral care composition.

In some embodiments, the disclosure provides for oral care compositions, e.g., any of Composition 1.0 et seq., where the oral care composition comprises a natural sweetener system comprising a combination of about 3% weight xylitol, and about 0.05% to about 3% weight of a natural sweetener system comprising Reb A 98% and GSG80 in a ratio from 1:1 to 6:1, e.g., 1:1, or 1:2, or 1:3, or, 1:4, or, 1:5, or 2:1, or 3:1, or 4:1, or 5:1, wherein the % weight are based on the total weight of the oral care composition.

In some embodiments, the disclosure provides for oral care compositions, e.g., any of Composition 1.0 et seq., where the oral care composition comprises a natural sweetener system comprising combination of 5.00% weight xylitol, and about 0.05% to about 3.00% weight of a natural sweetener system comprising Reb A 98% and GSG80 in a ratio from 1:1 to 6:1, e.g., 1:1, or 1:2, or 1:3, or, 1:4, or, 1:5, or 2:1, or 3:1, or 4:1, or 5:1, wherein the % weight are based on the total weight of the oral care composition.

In some embodiments, the disclosure provides for oral care compositions, e.g., any of Composition 1.0 et seq., where the oral care composition comprises a natural sweetener system comprising combination of 10% weight xylitol, and about 0.05% to about 3% weight of a natural sweetener system comprising Reb A 98% and GSG80 in a ratio from 1:1 to 6:1, e.g., 1:1, or 1:2, or 1:3, or, 1:4, or, 1:5, or 2:1, or 3:1, or 4:1, or 5:1, wherein the % weight are based on the total weight of the oral care composition.

In some embodiments, the disclosure provides for oral care compositions, e.g., any of Composition 1.0 et seq., where the oral care composition comprises a natural sweetener system comprising a combination of 5% weight xylitol, and about 0.10% weight of a natural sweetener system comprising Reb A 98% and GSG80 in a ratio (wt %) 1:1 to 6:1, e.g., 1:1, or 1:2, or 1:3, or, 1:4, or, 1:5, or 2:1, or 3:1, or 4:1, or 5:1, wherein the % weight are based on the total weight of the oral care composition.

In some embodiments, the disclosure provides for oral care compositions, e.g., any of Composition 1.0 et seq., where the oral care composition comprises a natural sweetener system comprising a ratio (wt %) Reb A to GSG80 1:1 to 6:1, e.g., 1:1, or 1:2, or 1:3, or, 1:4, or, 1:5, or 2:1, or 3:1, or 4:1, or 5:1, respectively (e.g., the natural sweetener system comprises Reb A 98% pure and GSG80 in a ratio (wt %) of 1:2) (e.g., a combination of xylitol, Reb A 98%, and GSG80) (e.g., a combination of 0.05% to 10% weight xylitol, Reb A 98%, and GSG80, wherein the ratio (wt %) Reb A to GSG80 is 1:2) (e.g., wherein Reb A 98% and GSG80 in a ratio (wt %) of 5:1) (e.g., wherein Reb A 98% and GSG80 are in a ratio (wt %) of 4:1.

In some embodiments, the disclosure provides for oral care compositions, e.g., any of Composition 1.0 et seq., comprising a natural sweetener system, wherein the natural sweetener system comprises Reb A 98% pure in an amount from about 0.01% to about 1.00% by weight based on the total weight of the composition.

In some embodiments, the disclosure provides for oral care compositions, e.g., any of Composition 1.0 et seq., comprising a natural sweetener system, wherein the natural sweetener system comprises Reb A 98% pure in an amount from 0.03% to 0.5% by weight based on the total weight of the composition.

In some embodiments, the disclosure provides for oral care compositions, e.g., any of Composition 1.0 et seq., comprising a natural sweetener system, wherein the natural sweetener system comprises Reb A 98% pure in about 0.75% by weight based on the total weight of the system.

In some embodiments, the disclosure provides for oral care compositions, e.g., any of Composition 1.0 et seq., comprising a natural sweetener system, wherein the natural sweetener system comprises Reb A 98% pure in about 0.72% by weight based on the total weight of the system.

In some embodiments, the disclosure provides for oral care compositions, e.g., any of Composition 1.0 et seq., comprising a natural sweetener system, wherein the natural sweetener system comprises Reb A 98% pure at about 0.30% by weight based on the total weight of the system.

In some embodiments, the disclosure provides for oral care compositions, e.g., any of Composition 1.0 et seq., comprising a natural sweetener system, wherein the natural sweetener system comprises GSG80 at about 0.15% by weight based on the total weight of the system.

In some embodiments, the disclosure provides for oral care compositions, e.g., any of Composition 1.0 et seq., comprising a natural sweetener system, wherein the natural sweetener system comprises GSG80 in about 0.18% by weight based on the total weight of the system.

In some embodiments, the disclosure provides for oral care compositions, e.g., any of Composition 1.0 et seq., comprising a natural sweetener system, wherein the natural sweetener system comprises GSG80 at about 0.60% by weight based on the total weight of the system.

In some embodiments, the disclosure provides for oral care compositions, e.g., any of Composition 1.0 et seq., comprising a natural sweetener system, wherein the natural sweetener system comprises GSG80 in an amount from 0.01% to 2% by weight based on the total weight of the composition.

In some embodiments, the disclosure provides for oral care compositions, e.g., any of Composition 1.0 et seq., comprising a natural sweetener system, wherein the natural sweetener system comprises GSG80 in an amount from 0.05% to 1% by weight based on the total weight of the system.

In some embodiments, the disclosure provides for oral care compositions, e.g., any of Composition 1.0 et seq., comprising a natural sweetener system, wherein the natural sweetener system comprises GSG80 in an amount of about 0.5% by weight based on the total weight of the system.

In some embodiments, the disclosure provides for oral care compositions, e.g., any of Composition 1.0 et seq., comprising a natural sweetener system, wherein the natural sweetener system comprises GSG95 in an amount from 0.01% to 2% by weight based on the total weight of the composition.

In some embodiments, the disclosure provides for oral care compositions, e.g., any of Composition 1.0 et seq., comprising a natural sweetener system, wherein the natural sweetener system comprises GSG95 in an amount from 0.05% to 1% by weight based on the total weight of the system.

In some embodiments, the disclosure provides for oral care compositions, e.g., any of Composition 1.0 et seq., comprising a natural sweetener system, wherein the natural sweetener system comprises GSG95 in an amount of about 0.50% by weight based on the total weight of the system.

In some embodiments, the disclosure provides for oral care compositions, e.g., any of Composition 1.0 et seq., comprising a natural sweetener system, wherein the natural sweetener system comprises GSG98 in an amount from about 0.01% to about 2% by weight based on the total weight of the composition.

In some embodiments, the disclosure provides for oral care compositions, e.g., any of Composition 1.0 et seq., comprising a natural sweetener system, wherein the natural sweetener system comprises GSG98 in an amount from 0.05% to 1% by weight based on the total weight of the system.

In some embodiments, the disclosure provides for oral care compositions, e.g., any of Composition 1.0 et seq., comprising a natural sweetener system, wherein the natural sweetener system comprises GSG98 in an amount of about 0.5% by weight based on the total weight of the system.

In some embodiments, the disclosure provides for oral care compositions, e.g., any of Composition 1.0 et seq., comprising a natural sweetener system, wherein the natural sweetener system comprises Reb A 98% pure in an amount of about 0.75% by weight and GSG80 in an amount of about 0.15%, wherein the wt. % is relative to the total weight of the system.

In some embodiments, the disclosure provides for oral care compositions, e.g., any of Composition 1.0 et seq., comprising a natural sweetener system, wherein the natural sweetener system comprises Reb A 98% pure in an amount of about 0.72% by weight and GSG80 in an amount of about 0.18% by weight based on the total weight of the system.

In some embodiments, the disclosure provides for oral care compositions, e.g., any of Composition 1.0 et seq., comprising a natural sweetener system, wherein the natural sweetener system comprises Reb A 98% pure in an amount of about 0.30% by weight and GSG80 in an amount of about 0.60%, by weight based on the total weight of the system.

### DETAILED DESCRIPTION OF THE INVENTION

It is to be understood that both the foregoing general description and the following detailed description are exemplary and explanatory only and are not intended to limit the invention, its application or uses.

As used herein, the use of the singular includes the plural unless specifically stated otherwise. As used throughout, ranges are used as shorthand for describing each and every value that is within the range. Any value within the range may be selected as the terminus of the range. As used herein, the use of a compound comprising several isomers or stereoisomers includes all the isomeric forms of that compound.

Unless otherwise specified, all percentages and amounts expressed herein and elsewhere in the specification should be understood to refer to percentages by weight of the entire composition. The amounts given are based on the active weight of the material.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Examples of such oral care compositions include, but are not limited to, toothpaste, dentifrice (e.g., toothpaste), mouthwash, mouth rinse, mouth spray, topical oral gel, denture cleanser, dental strips, beads, varnish, toothpaste tape, toothpaste film, toothpaste ribbon, dental floss, chewing gum, lozenges, gummies, tooth powder and the like.

As used herein, the term “toothpaste” refers to a paste, a gel, an emulsion, used on a toothbrush for cleaning the teeth. It is understood that a toothpaste for example will typically be diluted with water upon use, while a mouth rinse or mouth spray, typically will not be.

As used herein, the term “dentifrice” refers to a paste, gel, or liquid formulations unless otherwise specified. The dentifrice composition may be in any desired form such as deep striped, surface striped, multi-layered, having the gel surrounding the paste, or any combination thereof. Alternatively, the oral care composition may be dual phase dispensed from a separated compartment dispenser.

As used herein, the terms “mouthwash” or “mouth rinse” or “mouth spray” refer to oral care compositions that are substantially liquid in character, such as a spray, or rinse. In such a preparation, the orally acceptable carrier typically has an aqueous phase comprising water or a water and alcohol mixture. Further, in various embodiments, the oral care composition includes a humectant and surfactant as described below. Generally, the weight ratio of water to alcohol is in the range of 1:1 to 20:1, preferably 3:1 to 10:1 and more preferably 4:1 to 6:1. The total amount of water and alcohol mixture in this type of preparation is typically in an amount from 70.0% weight to 99.9% weight of the preparation. In various embodiments, the alcohol is typically ethanol or isopropanol.

As used herein, the term “topical oral gel” refers to a mixture prepared and immediately transferred into a retaining tray, such as those used in holding whitening gels, and the person may wear the tray for the effective period of time. The teeth that are in contact with the mixture will be treated. For use with retaining tray, the mixture may be in the form of a low-viscosity liquid or a gel. In certain embodiments, the whitening agent of the invention is formulated in an oral care composition comprising crosslinked copolymer of polyacrylic acid (sold under the name of Carbopol® polymer), glycerin and water.

As used herein, the term “varnish” refers to stock solution, or a mixture of stock solutions with water, applied to the teeth in a varnish formulation, wherein the varnish may stay on the tooth for an extended period of time for effective treatment. When the oral care composition is in the form of a gel or a varnish, then the composition of the present invention is a viscous liquid, preferably a gel, which maintains its consistency during storage, enabling the product to be painted on the tooth surface with a soft applicator pen or brush. Some embodiments provide a method utilizing an applicator to deliver the oral care composition, wherein the applicator is a pen and the pen is stored within an oral care implement. In some embodiments, the pen is removed from the oral care implement prior to application of the oral care composition to the tooth. In some embodiments, the composition is applied to the tooth after brushing. In some embodiments, the oral care composition is applied to the tooth after brushing with the oral care implement. In certain embodiments, the varnish comprises hydrophobic copolymers comprising at least one of octylacrylamide/acrylates/butylaminoethyl, methacrylate copolymer, VA/butyl maleate/isobornyl acrylate copolymer, acrylates/t-butylacrylamide copolymer, polyvinylpyrrolidone/vinyl acetate copolymer, triacontanyl polyvinylpyrrolidone copolymer, acrylates/dimethylaminoethyl methacrylate copolymer (sold under the name of Eudragit®), 2-propenoic acid 2-methyl-2-methylpropyl ester polymer with 2-propenoic acid and N-(1,1,3,3-tetramethylbutyl)-2-propenamide (sold under the name of DERMACRYL®), and/or combinations thereof.

As used herein, the terms “a denture cleanser” refers to a cleaner for dentures when they are out of the mouth.

As used herein, the terms “dental strips,” refer to disposable strips, which contain enamel safe oral care gel, placed directly on the teeth.

As used herein, the term “tooth powder” refers to a powder for cleaning the teeth.

As used herein, the term “actives,” refers to compounds that, when applied to a target tissue, provide a benefit or improvement to the target tissue. The actives may be delivered in the form of any oral care formulations, for example toothpaste, transparent paste, gel, mouthwash, powder, cream, dental strip, spray, gum, or any other known in the art. The term “active” and “agent” are used interchangeably in this disclosure. As used herein, “tooth” or “teeth” refers to natural teeth, dentures, dental plates, fillings, caps, crowns, bridges, dental implants, and the like, and any other hard surfaced dental prosthesis either permanently or temporarily fixed within the oral cavity. As used herein, the terms “orally acceptable carrier” refers to a material or combination of materials that are safe for use in the oral care compositions of the present invention. The nature of the orally acceptable carrier is specific to the form of the product. Orally acceptable carriers can comprise: abrasives, stain prevention compounds, erosion preventing agents, antitartar compounds, anticalculus compounds, polymer complexes, surfactants, humectants and/or combinations thereof. Further, orally acceptable carriers may comprise pH modifying agents, foam modulators, thickening agents, flavoring agents, antibacterial agents, desensitizing agents, anticariogenic agents, fluoride ion sources, anticalculus agents, and/or combinations thereof. The orally acceptable carriers comprise optionally nutrients, vitamins, antibacterial agents, zinc ion sources, fluoride ion sources, stannous ion sources and/or combinations thereof.

As used herein, the term “sweetness” refers to the basic taste commonly associated with eating rich foods in sugars, the sweet taste is pleasurable.

As used herein, the term “bitterness” refers to a sharp, pungent or disagreeable flavor, not associated with salty or sour taste.

As used herein, the term “off-taste” refers to an unwanted taste imparted by spoilage or contamination, a taste which is not natural.

As used here, “purity” or “pure” refers to the dry weight of the steviol glycoside, e.g. rebaudioside A, or enzymatically glucosylated steviol glycoside of interest, relative to the dry weight of mixture (e.g. an extract) containing the steviol glycoside or enzymatically glucosylated steviol glycoside, prior to preparation of the mixture.

As used herein, the terms “Reb-A” or “Rebaudioside-A” are used interchangeably and comprise all isomeric or diastereoisomeric forms thereof. For simplicity reasons, the terms “enzymatically glucosylated steviol glycosides extract” will be referred to as “GSG” and will be used interchangeably. As used herein, the terms “enzymatically glucosylated steviol glycosides extract” or “GSG” comprise all isomeric or diastereoisomeric forms thereof. The oral care compositions of the invention may comprise various agents which are active to protect and enhance the strength and integrity of the enamel and tooth structure and/or to reduce bacteria and associated tooth decay and/or gum diseases. Effective concentration of the active ingredients used herein will depend on the particular agent and the delivery system used.

Sweeteners

In certain embodiments the disclosure provides for oral care compositions comprising a natural sweetener system wherein the natural sweetener system comprises a Reb A extract and GSG extract. In some embodiments the purity of the Reb A extract is in a range of from 95% to 99% by wt., relative to the total weight of the Reb A extract. In yet a further embodiment, the purity of the Reb A extract in the oral care compositions described herein is 95% pure, or 96% pure, or 97% pure, or 98% pure, or 99% pure, relative to the total weight of the Reb extract. In some embodiments the Reb A in the oral care compositions described herein is of 98% pure, relative to the total weight of the Reb A extract.

In some embodiments the content in GSG within the extract is from 80% to 98% by weight, relative to the weight of the GSG extract. In some embodiments, the content of GSG in the extract is 80% by weight, the wt. % relative to the total weight of the extract. In further embodiments the content of the GSG in the extract may be 85% by weight, the wt. % relative to the total weight of the extract. In some embodiments the content of GSG in the extract may be 90% by weight, the wt. % relative to the total weight of the extract. In further embodiments the content of GSG in the extract may be 95% by weight, the wt. % relative to the total weight of the extract. In still other embodiments, the content of GSG in the extract may be 98% by weight, the wt. % relative to the total weight of the extract.

An extract of GSG, wherein the content in enzymatically glucosylated steviol glycosides extract is 80% by weight relative to the total weight of the GSG extract, will be referred to as GSG80. The extract of GSG, wherein the content in enzymatically glucosylated steviol glycosides extract is 95% by weight relative to the total weight of the extract, will be referred to as GSG95. The number following “GSG” refers to the content in % by weight in the enzymatically glucosylated steviol glycosides extract within the GSG extract.

In some embodiments, the natural sweetener system of the invention comprises a combination of Reb A 98% pure extract and GSG80.

In some embodiments, the oral care composition comprises a natural sweetener system comprising Rebaudioside A extract and an enzymatically glucosylated steviol glycosides extract; an orally acceptable carrier; wherein Rebaudioside A extract has a purity of from 95% to 99% by wt., relative to the total weight of the extract; and the enzymatically glucosylated steviol glycosides extract has a content in enzymatically glucosylated steviol glycosides extract from 80% by weight to 98% by weight, the wt. % relative to the total weight of the extract.

In some embodiments, the natural sweetener system of the disclosure comprises a combination of Rebaudioside A extract that has a purity from 95% to 99% by wt., the wt. % relative to the total weight of the extract, and GSG95. In some embodiments, the natural sweetener system has a ratio (wt %) of Reb A to GSG95 of 1:1, or 1:2, or 1:3, or, 1:4, or, 1:5, or 2:1, or 3:1, or 4:1, or 5:1, respectively. In some embodiments the natural sweetener system comprises Reb A 98% pure and GSG95 in a ratio of 1:2.

In some embodiments, the natural sweetener system of the invention comprises a combination of xylitol, Reb A 98% by wt. pure extract, the wt. % relative to the total weight of the extract, and GSG95.

In some embodiments, the natural sweetener system of the invention comprises a combination of xylitol, Reb A 98% by wt. pure extract, the wt. % relative to the total weight of the extract, and GSG95, wherein the wt % ratio of Reb A to GSG95 is 1:2.

In some embodiments, the natural sweetener system of the invention is present from about 0.05% to about 3.0% weight in the oral care composition, wherein the % weight is based on the total weight of the oral care composition. In some embodiments, the natural sweetener system of the invention is present from about 0.05% to about 3.0% weight in the oral care composition, wherein the wt % ratio of Reb A extract to GSG is 1:1, or 1:2, or 1:3, or, 1:4, or, 1:5, or 2:1, or 3:1, or 4:1, or 5:1, respectively.

In some embodiments, the natural sweetener system of the invention is present from about 0.05% to about 3% weight in the oral care composition, wherein the ratio of Reb A extract to GSG80 is 1:1, or 1:2, or 1:3, or, 1:4, or, 1:5, or 2:1, or 3:1, or 4:1, or 5:1, respectively.

In some further embodiments, the natural sweetener system of the invention is present from about 0.05% to about 3.00% weight in the oral care composition, wherein the ratio of Reb A 98% by wt. pure extract, the wt. % relative to the total weight of the extract, to GSG95 is 1:1, or 1:2, or 1:3, or, 1:4, or, 1:5, or 2:1, or 3:1, or 4:1, or 5:1, respectively.

In some embodiments, the natural sweetener system of the invention is present from about 0.05% to about 3.0% weight in the oral care composition, wherein the ratio of Reb A 98% by wt. pure extract, the wt. % relative to the total weight of the extract, to GSG98 is 1:1, or 1:2, or 1:3, or, 1:4, or, 1:5, or 2:1, or 3:1, or 4:1, or 5:1, respectively.

In some embodiments, the disclosure provides for oral care compositions comprising a natural sweetener system, as described herein in an amount of about 0.10%, or about 0.20%, or about 0.30%, or about 0.40%, or about 0.50%, or about 0.60%, or about 0.70%, or about 0.80%, or about 0.90%, or about 1.00%, or about 1.10%, or about 1.20%, or about 1.30%, or about 1.40%, or about 1.50%, or about 1.60%, or about 1.70%, or about 1.80%, or about 1.90%, or about 2.00%, or about 2.10%, or about 2.20%, or about 2.30%, or about 2.40%, or about 2.50%, or about 2.60%, or about 2.70%, or about 2.80%, or about 2.90% or about 3.00% by weight relative to the weight of the oral care composition.

In some embodiments, the natural sweetener system of the disclosure comprises a combination of about 0.05% to about 10% weight xylitol, and about 0.05% to about 3% weight of the natural sweetener system of the disclosure, wherein the % weight are based on the total weight of the oral care composition.

In some embodiments, the natural sweetener system of the invention comprises a combination of about 3% weight xylitol, and about 0.05% to about 3% weight of a natural sweetener system comprising a Reb A 98% by wt. pure extract, the wt. % relative to the total weight of the extract, and GSG80 in a ratio of 1:1, or 1:2, or 1:3, or, 1:4, or, 1:5, or 2:1, or 3:1, or 4:1, or 5:1, wherein the % weight are based on the total weight of the oral care composition.

In some embodiments, the disclosure provides an oral care composition comprising a natural sweetener system of the invention comprises a combination of 5% weight xylitol, and about 0.05% to about 3% weight of a natural sweetener system, wherein the % weight are based on the total weight of the oral care composition, wherein the natural sweetener system comprises a Reb A 98% by wt. pure extract, the wt. % relative to the total weight of the extract, and GSG80 in a ratio of 1:1, or 1:2, or 1:3, or, 1:4, or, 1:5, or 2:1, or 3:1, or 4:1, or 5:1.

In some embodiments, the natural sweetener system of the invention comprises a combination of 10.00% weight xylitol, and about 0.05% to about 3.00% weight of a natural sweetener system comprising Reb A 98% by wt. pure extract, the wt. % relative to the total weight of the extract, and GSG80 in a ratio of 1:1, or 1:2, or 1:3, or, 1:4, or, 1:5, or 2:1, or 3:1, or 4:1, or 5:1, wherein the % weights are based on the total weight of the oral care composition.

In some embodiments, the natural sweetener system of the invention comprises a combination of 5.00% weight xylitol, and about 0.10% weight of a natural sweetener system comprising Reb A 98% by wt. pure extract, the wt. % relative to the total weight of the extract, and GSG80 in a ratio of 1:1, or 1:2, or 1:3, or, 1:4, or, 1:5, or 2:1, or 3:1, or 4:1, or 5:1, wherein the % weight are based on the total weight of the oral care composition.

In some embodiments, the natural sweetener system has a ratio Reb A to GSG80 of 1:1, or 1:2, or 1:3, or, 1:4, or, 1:5, or 2:1, or 3:1, or 4:1, or 5:1, respectively.

In some embodiments the natural sweetener system comprises Reb A 98% by wt. pure extract, the wt. % relative to the total weight of the extract, and GSG80 in a ratio of 1:2. In some embodiments, the natural sweetener system of the invention comprises a combination of xylitol, Reb A 98% by wt. pure extract, the wt. % relative to the total weight of the extract, and GSG80.

In some embodiments, the natural sweetener system of the disclosure comprises a combination of 0.05% to 10% weight xylitol, Reb A 98% by wt. pure extract, the wt. % relative to the total weight of the extract, and GSG80, wherein the ratio Reb A to GSG80 is 1:2. In some embodiments the natural sweetener system comprises Reb A 98% by wt. pure extract, the wt. % relative to the total weight of the extract, and GSG80 in a ratio of 5:1. In some embodiments the natural sweetener system comprises Reb A 98% by wt. pure extract, the wt. % relative to the total weight of the extract, and GSG80 in a ratio of 4:1.

In certain embodiments, the natural sweetener system described herein is free from artificial sweeteners, wherein the artificial sweetener is selected from: saccharin, sucralose, aspartame, sodium cyclamate, acesulfame K, advantame, and/or neotame.

In certain embodiments, the natural sweetener system of the invention provides unique features, such as enhanced organoleptic properties, to oral care compositions.

In further embodiments, the disclosure provides a method to improve oral health comprising applying an effective amount of an oral care composition of the disclosure, wherein the oral care composition comprises a natural sweetener system (e.g., where the natural sweetener system comprises a Reb A extract and GSG extract) to the oral cavity of a subject in need thereof.

In various embodiments, the disclosure provides an oral care composition comprising a natural sweetener system comprising a Reb A extract and GSG extract, wherein the oral care composition is free from bitter or metallic taste.

In various embodiments, the disclosure provides an oral care composition comprising a natural sweetener system, wherein the system comprises a Reb A extract and GSG extract, and wherein the oral care composition also comprises one or more orally acceptable carrier materials.

Some embodiments of the present disclosure provide an oral care composition comprising a natural sweetener system comprising a Reb A extract and GSG extract and, wherein the oral care composition comprises one or more: abrasives, stain prevention compounds, erosion preventing agents, antitartar compounds, anticalculus compounds, polymer complexes, surfactants, humectants and/or combinations thereof.

Some embodiments of the present disclosure provide an oral care composition comprising a natural sweetener system comprising a Reb A extract and GSG extract and, wherein the oral care composition comprises one or more: pH modifying agents, foam modulators, thickening agents, flavoring agents, antibacterial agents, desensitizing agents, anticariogenic agents, fluoride ion sources, anticalculus agents, and/or combinations thereof.

Some embodiments of the present disclosure provide an oral care composition comprising a natural sweetener system comprising a Reb A extract and GSG extract and wherein the oral care composition comprises one or more: nutrients, vitamins, and/or antibacterial agents.

Some embodiments of the present disclosure provide an oral care composition comprising a natural sweetener system comprising a Reb A extract and GSG extract and wherein the oral care composition comprises one or more abrasives. In certain embodiments the abrasives are selected from: silica gel, perlite, hydrated silica, high cleaning silica, core shell silica, precipitated silica, calcium carbonate, and/or calcium pyrophosphate.

Some embodiments of the present invention provide an oral care composition comprising a natural sweetener system comprising a Reb A extract and GSG extract, wherein the oral care composition is in the form selected from: toothpaste, dentifrice, mouthwash, mouth spray, mouth rinse, mouth spray, topical oral gel and denture cleanser.

Some embodiments of the present invention provide an oral care composition comprising a natural sweetener system comprising a Reb A extract and GSG extract, wherein the oral care composition is in the form selected from: dental strips, beads, varnish, toothpaste tape, toothpaste film, toothpaste ribbon, dental floss, tablets, chewing gum, gummies, lozenges and toothpowder.

Some embodiments of the present disclosure provide an oral care composition comprising a natural sweetener system, wherein the sweetener system comprises a Reb A extract and a GSG extract, and wherein the oral care composition comprises a fluoride ion source (e.g., sodium fluoride or stannous fluoride).

Some embodiments of the present disclosure provide an oral care composition comprising a natural sweetener system, wherein the sweetener system comprises a Reb A extract and a GSG extract, and wherein the oral care composition comprises a zinc ion source (e.g., zinc citrate, zinc oxide and/or zinc phosphate).

Some embodiments of the present disclosure provide an oral care composition comprising a natural sweetener system, wherein the sweetener system comprises a Reb A extract and a GSG extract, and wherein the oral care composition comprises a stannous ion source (e.g., stannous fluoride, stannous pyrophosphate and/or stannous chloride).

In some embodiments the disclosure provides an oral care composition comprising about 0.05% to about 3% by weight of the natural sweetener system, wherein the sweetener system comprises a Reb A extract and GSG extract, wherein the weight % is based on the total weight of the composition.

In some embodiments the disclosure provides an oral care composition comprising about 0.1% by weight of a natural sweetener system, wherein the sweetener system comprises a Reb A extract and a GSG extract, and wherein the weight % is based on the total weight of the composition.

In some embodiments the disclosure provides an oral care composition comprising about 0.9% by weight of a natural sweetener system, wherein the sweetener system comprises a Reb A extract and a GSG extract, and wherein the weight % is based on the total weight of the composition.

In some embodiments the disclosure provides an oral care composition comprising about 1% by weight of a natural sweetener system, wherein the sweetener system comprises a Reb A extract and a GSG extract, and wherein the weight % is based on the total weight of the composition.

In some embodiments the disclosure provides an oral care composition comprising about 1.5% by weight of a natural sweetener system, wherein the sweetener system comprises a Reb A extract and a GSG extract, and wherein the weight % is based on the total weight of the composition.

In some embodiments the disclosure provides an oral care composition comprising about 0.1% to 1% by weight of a natural sweetener system, wherein the sweetener system comprises a Reb A extract and a GSG extract, and wherein the weight % is based on the total weight of the composition. In some embodiments the natural sweetener system comprises Reb A 98% by wt. pure extract, the wt. % relative to the total weight of the extract, in an amount from about 0.03% to about 0.50% by weight based on the total weight of the system. In some embodiments the natural sweetener system comprises Reb A 98% by wt. pure extract, the wt. % relative to the total weight of the extract, in about 0.75% by weight based on the total weight of the system. In some embodiments the natural sweetener system comprises Reb A 98% by wt. pure extract, the wt. % relative to the total weight of the extract, in about 0.72% by weight based on the total weight of the system. In some embodiments the natural sweetener system comprises Reb A 98% by wt. pure extract, the wt. % relative to the total weight of the extract, in about 0.30% by weight based on the total weight of the system.

In some embodiments the disclosure provides an oral care composition comprising about 0.1% to 1% by weight of a natural sweetener system, wherein the sweetener system comprises a Reb A extract and a GSG extract, and wherein the weight % is based on the total weight of the composition.

In some embodiments the disclosure provides an oral care composition comprising a natural sweetener, wherein the natural sweetener comprises GSG80 in an amount about 0.15% by weight based on the total weight of the system.

In some embodiments the disclosure provides an oral care composition comprising a natural sweetener, wherein the natural sweetener comprises GSG80 in an amount about 0.18% by weight based on the total weight of the system.

In some embodiments the disclosure provides an oral care composition comprising a natural sweetener, wherein the natural sweetener comprises GSG80 in an amount about 0.60% by weight based on the total weight of the system.

In some embodiments the disclosure provides an oral care composition comprising a natural sweetener, wherein the natural sweetener comprises GSG80 in an amount from about 0.02% to about 2.00% by weight based on the total weight of the system.

In some embodiments the disclosure provides an oral care composition comprising a natural sweetener, wherein the natural sweetener comprises GSG80 in an amount about from about 0.05% to about 1.00% by weight based on the total weight of the system.

In some embodiments the disclosure provides an oral care composition comprising a natural sweetener, wherein the natural sweetener comprises GSG80 in an amount about 0.50% by weight based on the total weight of the system.

In some embodiments the disclosure provides an oral care composition comprising a natural sweetener, wherein the natural sweetener comprises GSG95 in an amount from about 0.02% to about 2.00% by weight based on the total weight of the system.

In some embodiments the disclosure provides an oral care composition comprising a natural sweetener, wherein the natural sweetener comprises GSG95 in an amount from about 0.05% to about 1% by weight based on the total weight of the system.

In some embodiments the disclosure provides an oral care composition comprising a natural sweetener, wherein the natural sweetener comprises GSG95 in an amount of about 0.50% by weight based on the total weight of the system.

In some embodiments the disclosure provides an oral care composition comprising a natural sweetener, wherein the natural sweetener comprises GSG98 in an amount from about 0.02% to about 2.00% by weight based on the total weight of the system.

In some embodiments the disclosure provides an oral care composition comprising a natural sweetener, wherein the natural sweetener comprises GSG98 in an amount from about 0.05% to about 1.00% by weight based on the total weight of the system.

In some embodiments the disclosure provides an oral care composition comprising a natural sweetener, wherein the natural sweetener comprises GSG98 in an amount of about 0.50% by weight based on the total weight of the system.

In some embodiments the disclosure provides an oral care composition comprising a natural sweetener, wherein the natural sweetener system comprises Reb A 98% by wt. pure extract (relative to the total weight of the extract) in an amount of 0.2%-2% by weight (relative to the total wt. % of the system) and GSG80 in an amount of about 0.05%-1% by weight (relative to the total wt. % of the system).

In some embodiments the disclosure provides an oral care composition comprising a natural sweetener, wherein the natural sweetener system comprises Reb A 98% by wt. pure extract (relative to the total weight of the extract) in an amount of about 0.75% by weight (relative to the total wt. % of the system) and GSG80 in an amount of about 0.15% by weight (relative to the total wt. % of the system).

In some embodiments the disclosure provides an oral care composition comprising a natural sweetener, wherein the natural sweetener system comprises Reb A 98% by wt. pure extract (relative to the total weight of the extract) in an amount of about 0.72% by weight (relative to the total wt. % of the system) and GSG80 in an amount of about 0.18% by weight (relative to the total wt. % of the system).

In some embodiments the disclosure provides an oral care composition comprising a natural sweetener, wherein the natural sweetener system comprises Reb A 98% by wt. pure extract (relative to the total weight of the extract) in an amount of about 0.3% by weight (relative to the total wt. % of the system) and GSG80 in an amount of about 0.6% by weight (relative to the total wt. % of the system).

Oral Care Composition Ingredients

In some embodiments, the oral care compositions disclosed herein may comprise at least one abrasive agent. The role of an abrasive is to act as a polishing agent within the oral care composition. In some embodiments, the abrasive agents include without limitation silica (in the form of silica gel, hydrated silica, high cleaning silica, perlite, core shell silica or precipitated silica), alumina, insoluble phosphates, calcium carbonate, resinous abrasives such as urea-formaldehyde condensation products, dicalcium phosphate, calcium pyrophosphate, calcium carbonate, sodium bicarbonate, alumina, dicalcium orthophosphate dihydrate, n-calcium pyrophosphate, tricalcium phosphate, calcium polymetaphosphate, insoluble sodium polymetaphosphate, precipitate calcium carbonate. The average particle size of the abrasive is generally about 0.1 μm to about 30 μm for example about 1 μm to about μm or about 5 μm to about 15 μm. In some embodiments, one or more abrasives are present in an amount of about 0.1% to about 50% by weight based on the total weight of the composition. In some embodiments, the abrasive is calcium pyrophosphate. In some embodiments, the calcium pyrophosphate is present in an amount from about 5% to about 50% by weight based on the total weight of the composition. In some embodiments the abrasive is core shell silica. In some embodiments the abrasive is precipitated calcium carbonate. In some embodiments the abrasive is perlite, sold under the name of Perlite ImerCare® 25P. In some embodiments the abrasive is core shell silica. In some embodiments the abrasive is precipitated calcium carbonate. In some embodiments the oral care composition of the invention comprises an abrasive reducing agent comprising cellulose, such as non-colloidal microcrystalline cellulose, colloidal microcrystalline cellulose and/or combinations thereof.

In some embodiments, the oral care compositions disclosed herein may comprise at least one whitening agent. In some embodiments the whitening agents include peroxides and hydroperoxides, such as hydrogen peroxide, peroxides of alkali and alkaline earth metals, organic peroxy compounds, peroxy acids, peroxymonosulfates, pharmaceutically-acceptable salts thereof, vitamin C derivatives and/or combinations thereof. In some embodiments the whitening agents include non-peroxy compounds, such as chlorine dioxide, chlorites, hypochlorites, ascorbic acid derivatives, titanium dioxide and hydroxyapatite. In some embodiments the whitening agent is a peroxide complex comprising crosslinked polyvinylpyrrolidone complexed with hydrogen peroxide. In some embodiments, the whitening agent is present in an amount of about 0.1% to about 6.0% by weight based on the total weight of the composition. In some embodiments the whitening agents are vitamin C derivatives comprising L-ascorbic acid, calcium ascorbate, calcium 1-ascorbate dihydrate, magnesium ascorbate, potassium ascorbate, magnesium L-ascorbyl phosphate, L-ascorbic acid 2-phosphate sesquimagnesium salt hydrate, sodium ascorbate, dehydro-1-(+)-ascorbic acid dimer, sodium ascorbyl phosphate, ascorbic acid-2-glucoside, ascorbyl dipalmitate, ascorbyl methylsilanol pectinate, ascorbyl stearate, disodium ascorbyl sulfate, ascorbyl 6-palmitate and/or combinations thereof.

In some embodiments, the oral care compositions disclosed herein may comprise at least one stain prevention agent. In some embodiments the stain prevention agents include tetrasodium pyrophosphate (TSPP), disodium pyrophosphate (SAPP), sodium tripyrophosphate (STPP), sodium hexametaphosphate (SHMP), zinc citrate and/or combinations thereof. In some embodiments, one or more stain prevention agents are present in an amount of about 0.1% to about 60.0% by weight based on the total weight of the composition.

In some embodiments, the oral care compositions disclosed herein may comprise at least one surfactant agent. The role of the surfactant is to enhance the stability of the oral care composition and create foam during stirring which ensures cleaning the oral cavity. In some embodiments the surfactant agents comprise anionic, nonionic or amphoteric surfactants. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium cocoyl monoglyceride sulfonate, sodium stearate, sodium lauryl sarcosinate (sold under the name of Maprosyl® 30-B), lauryl glucoside, sodium lauryl isoethionate (sold under the name of Tauranol® WHSP), sodium laureth carboxylate, sodium methyl cocoyl taurate, sodium lauryl sulfate (same as sodium dodecyl sulfate or SLS or SDS) and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation derivatives of C₈₋₂₀ aliphatic secondary and tertiary amines having an anionic group such as carboxylate, sulfate, sulfonate, phosphate, phosphonate or cocoamidopropyl betaine, C₁₀₋₁₆ alkyl glucosides, alkyl betaines and alkyl amido betaines. In some embodiments the surfactant is a mixture of sodium cocoamphoacetate and glycerin and lauryl glucoside and sodium cocoyl glutamate, sold under the name of Plantapon®, and sodium lauryl glucose carboxylate. In some embodiments the surfactant is sodium carboxymethyl cellulose, sodium stearate, lauryl glucoside, polyanionic cellulose, sodium lauryl isoethionate, sodium lauryl sarcosinate, sodium cocoyl glutamate, disodium cocoyl glutamate, cocamidopropyl betaine, and/or combinations thereof. In some embodiments, one or more surfactants may be present in a total amount of from about 0.10% to about 10.00% by weight based on the total weight of the composition. In some embodiments, one or more surfactants may be present in a total amount of from about 1.80% to about 3.00% by weight based on the total weight of the composition. In some embodiments, one or more surfactants may be present in a total amount of from about 1.90% to about 2.70% by weight based on the total weight of the composition. In some embodiments, one or more surfactants may be present in a total amount of about 2.60% by weight based on the total weight of the composition.

In some embodiments, the oral care compositions disclosed herein may comprise at least one thickening agent. In some embodiments the thickening agents include without limitation carbomers, also known as carboxyvinyl polymers, carrageenans, also known as Irish moss and more particularly carrageenan (iota-carrageenan), high molecular weight polyethylene glycols (such as CARBOWAX® available from The Dow Chemical Company), cellulosic polymers such as hydroxyethylcellulose, carboxymethyl cellulose (CMC) and salts thereof, CMC sodium, natural gums such as karaya, xanthan gum, arabic and tragacanth, colloidal magnesium aluminum silicate, and colloidal silica, fumed silica, microcrystalline cellulose and/or combinations thereof. In some embodiments, the thickening agent is present in a total amount of about 0.1% to about 90.0% by weight based on the total weight of the composition. In some embodiments, the one or more optional thickening agents are present in a total amount of about 1% to about 50% by weight based on the total weight of the composition. In some embodiments, the one or more optional thickening agents are present in a total amount of about 5% to about 35% by weight based on the total weight of the composition.

In some embodiments, the oral care compositions disclosed herein may comprise at least one humectant agent. In some embodiments the humectant agents include polyhydric alcohols such as vegetable glycerin, non crystal sorbitol, xylitol or low molecular weight polyethylene glycols (PEGs) or polyoxyethylenes. High molecular weight PEGs are suitable, including those having an average molecular weight of about 200,000 to about 7,000,000, for example about 500,000 to about 5,000,000 or about 1,000,000 to about 2,500,000. One or more PEGs are optionally present in a total amount of about 0.1% by weight to about 10.0% by weight, for example about 0.20% by weight to about 5.00% by weight or about 0.25% by weight to about 2.00% by weight based on the total weight of the composition.

In some embodiments, the oral care compositions disclosed herein may comprise at least one adhesion agent. The role of the adhesion agent is to ensure the retention of the oral care composition on the oral cavity surface. In some embodiments, the adhesion agents include adhesives, film forming materials, viscosity enhancers, hydrophilic organic polymers, hydrophobic organic polymers, silicone gums, silicone adhesives, silicas, and/or combinations thereof. Adhesion agents are preferably present at a level of from about 0.01% by weight to about 75.00% by weight, optionally from about 1.00% by weight to about by weight based on the total weight of the composition.

In some embodiments, the oral care compositions disclosed herein may comprise at least one hydrophobic polymer carrier, wherein the composition is free of water. The term hydrophobic or water-insoluble as applied to polymers and as employed herein refers to polymers which are substantially non-aqueous having a water solubility of less than one gram per 100 grams of water at 25° C. In certain embodiments, the at least one hydrophobic polymer carrier is a silicone pressure sensitive adhesive. In certain embodiments, the at least one hydrophobic polymer is selected for the carrier to produce a tooth whitening composition having a viscosity ranging from about 1,000 cPs to about 900,000 cPs, such as from about 10,000 cPs to about 900,000 cPs or from about 10,000 cPs to about 100,000 cPs. One class of hydrophobic polymers that may be used according to certain exemplary embodiments comprises siloxane polymers, which are also generally known in the art as “silicone” polymers, such as silicone pressure sensitive adhesives (PSA). In certain embodiments disclosed herein, the hydrophobic polymers in the carrier are those in which a whitening agent can be dispersed and are well known in the art. Many such silicone polymers are commercially available. In various embodiments, a silicone-based hydrophobic polymer is a polyorganosiloxane, such as polydimethylsiloxane. The oral care compositions disclosed herein may further comprise crospovidone (poly[N-vinyl-2-pyrrolidone]) as an adhesion enhancing agent. Crospovidone may be present in the oral care composition in an amount ranging from about 10% by weight to about 30%, by weight or from about 15% by weight to about 25% by weight, or from about 18% by weight to about 25% weight based on the total weight of the composition. The oral care compositions disclosed herein may further comprise N-vinyl heterocyclic polymers. The N-vinyl heterocyclic polymers are derived from N-heterocyclic vinyl monomers, or are specific monomers such as N-vinyl imides or N-vinyl lactams. One preferred polymer is poly-N-vinyl-poly-2-pyrrolidone (PVP).

In some embodiments, the oral care compositions disclosed herein may comprise at least one antioxidant agent. In some embodiments, the antioxidant agents include butylated hydroxyanisole (BHA), butylated hydroxytoluene (BHT), vitamin A, carotenoids, quercetin, rutin, catechin, trolox, vitamin E, flavonoids, polyphenols, ascorbic acid, 2,4-dihydroxybenzoic acid, m-methoxybenzoic acid, m-hydroxybenzoic acid, p-hydroxybenzoic acid, 3,4-dihydroxybenzoic acid, stannous compounds, potassium or sodium meta-bisulfite, ammonium sulfate, herbal antioxidants, chlorophyll, melatonin, and/or combinations thereof.

In some embodiments, the oral care compositions disclosed herein may comprise erosion prevention agents. Sodium fluoride, arginine, arginine phosphate, arginine hydrochloride, arginine bicarbonate, sodium monofluorophosphate, potassium citrate, titanium phosphate, zinc oxide, zinc citrate, zinc citrate trihydrate, zinc lysine chloride complexes, zinc arginine chloride complexes are erosion prevention agents. L-arginine is an erosion preventing agent of choice.

In some embodiments, the oral care compositions disclosed herein may comprise one or more preservative agents. In some embodiments, the preservation agent is selected from: natural benzyl alcohol, chlorhexidine, benzoic acid, benzoic acid salts, quaternary ammonium compounds, benzalkonium chloride and combinations thereof.

In some embodiments, the oral care compositions disclosed herein may comprise one or more antimicrobial agents. In some embodiments, the antimicrobial agents may include triclosan (5-chloro-2-(2,4-dichlorophenoxy)phenol); 8-hydroxyquinoline and salts thereof; zinc and stannous ion sources such as zinc citrate, zinc sulphate, zinc glycinate, sodium zinc citrate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides, cetylpyridinium chloride (CPC), combinations of CPC with zinc and/or enzymes, tetradecylpyridinium chloride, and N-tetradecyl-4-ethylpyridinium chloride; bisguanides, such as chlorhexidine digluconate, hexetidine, octenidine, and alexidine; halogenated bisphenolic compounds such as 2,2′ methylene-bis-(4-chloro-6-bromophenol); benzalkonium chloride; salicylanilide, domiphen bromide; iodine; sulfonamides; bisbiguanides; phenolics; piperidine derivatives such as delmopinol and octapinol; magnolia extract; grapeseed extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin, clindamycin, and combinations thereof.

In some embodiments, the oral care compositions disclosed herein may comprise at least one tartar control (anticalculus) agent. In some embodiments, the tartar control agents can include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1, 1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and/or combinations thereof, wherein sodium may optionally be replaced by potassium or ammonium. Anticalculus agents include polycarboxylate polymers and polyvinyl methyl ether/maleic anhydride (PVME/MA) copolymers, such as those available under the Gantrez™ brand from ISP, Wayne, N.J. One or more anticalculus agents are optionally present in an anticalculus effective total amount, typically about 0.01% by weight to about 50% by weight, for example about 0.05% by weight to about 25% by weight or about 0.1% by weight to about 15% by weight based on the total weight of the composition.

In some embodiments, the oral care compositions disclosed herein may comprise a stannous ion source. Stannous ions may act as a periodontal active, tartar control agent, anticariogenic agent or tooth desensitizer. In some embodiments, the stannous ion source is selected from: stannous fluoride, other stannous halides such as stannous chloride dihydrate, organic stannous carboxylate salts such as stannous formate, acetate, gluconate, lactate, tartrate, oxalate, malonate and citrate, stannous ethylene glyoxide, and/or combinations thereof.

In some embodiments, the oral care compositions disclosed herein may comprise a fluoride ion source. In some embodiments, the fluoride ion source is selected from potassium, sodium and ammonium fluorides, stannous fluoride, sodium monofluorophosphates, monofluorophosphates, indium fluoride, and/or combinations thereof.

In some embodiments, the oral care compositions disclosed herein may comprise a zinc ion source. In some embodiments the zinc ion source may act as malodor neutralization agents. In certain embodiments, the zinc ion sources is selected from: zinc oxide, zinc citrate, zinc chloride, zinc acetate, zinc lactate, zinc salicylate, zinc sulfate, zinc phosphate, zinc tartrate and zinc nitrate and combinations thereof.

In some embodiments, the oral care compositions disclosed herein may comprise a colorant source. In some embodiments, the colorant is selected from: pigments, dyes, lakes, agents imparting a particular luster or reflectivity such as pearling agents, and combinations thereof. In various embodiments, colorants are operable to provide a white or light-colored coating on a dental surface, to act as an indicator of locations on a dental surface that have been effectively contacted by the oral care composition, and/or to modify appearance, in particular color and/or opacity, of the composition to enhance attractiveness to the consumer.

In some embodiments, the oral care compositions disclosed herein may comprise a flavoring agent. In some embodiments, the flavoring agents include any orally acceptable natural or synthetic flavoring agent may be used, such as flavoring oils, flavoring aldehydes, esters, alcohols, similar materials, and/or combinations thereof. Flavoring agents may also include vanillin, sage, marjoram, parsley oil, spearmint oil, cinnamon oil, oil of wintergreen (methyl salicylate) peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, bean- and nut-derived flavors such as coffee, cocoa, cola, peanut, almond, adsorbed and encapsulated flavoring agents and/or combinations thereof.

In some embodiments, the oral care compositions disclosed herein may comprise a mouth-feel agent. In some embodiments, the mouth-feel agents include materials imparting a desirable texture or other feeling during use of the composition of the invention.

In some embodiments, the oral care compositions disclosed herein may comprise a pH modifying agent. The role of the pH modifying agent is to keep the oral care composition at an orally acceptable pH range. In some embodiments, the pH modifying agents include acidifying agents to lower pH, basifying agents to raise pH, and buffering agents to control pH within a desired range. In some embodiments the compositions described herein comprise orally acceptable pH modifying agents selected from: carboxylic, phosphoric and sulfonic acids, acid salts, monosodium citrate, disodium citrate, monosodium malate, alkali metal hydroxides such as sodium hydroxide, carbonates such as sodium carbonate, bicarbonates, sesquicarbonates, borates, silicates, phosphates, monosodium phosphate, trisodium phosphate, pyrophosphate salts, imidazole, and/or combinations thereof.

In some embodiments, the oral care compositions disclosed herein may comprise a viscosity modifier. The role of the viscosity modifier is to ensure the oral care composition is homogenous. In some embodiments, the oral care compositions described herein comprise a viscosity modifier selected from: oral mineral oil, petrolatum, clays and organomodified clays, silica, and combinations thereof.

In some embodiments, the oral care compositions disclosed herein may comprise a foam modulator. The role of the foam modulator is to generate foam upon mixing which contributes to the dispersion of the oral care composition in the oral cavity. In some embodiments, the foam modulators include sodium lauroyl sarcosinate, sodium lauryl sulfate powder, sodium lauryl sulfate liquid, polyethylene glycols (PEGs), also known as polyoxyethylenes. The oral care composition of the invention may comprise a saliva stimulating source. The role of the saliva stimulating agent is to keep the mouth from drying. In some embodiments, the saliva stimulating source includes food acids such as citric, lactic, rilalic, succinic, ascorbic, adipic, fumaric and tartaric acids, and/or combinations thereof.

In some embodiments, the oral care compositions disclosed herein may comprise a breath freshening agent. In some embodiments, the breath freshening agent includes zinc salts such as zinc gluconate, zinc citrate and zinc chlorite, a-ionone, and/or combinations thereof. The oral care composition of the invention may comprise a desensitizing agent. In some embodiments, the desensitizing agents include potassium citrate, potassium chloride, potassium tartrate, potassium bicarbonate, potassium oxalate, potassium nitrate, strontium salts, and/or combinations thereof.

In some embodiments, the oral care compositions disclosed herein may comprise a nutrient agent. In some embodiments, the nutrient includes vitamins, minerals, amino acids, and/or combinations thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and/or combinations thereof. Nutritional supplements include amino acids, arginine, L-tryptophane, L-lysine, methionine, threonine, levocarnitine and L-carnitine, lipotropics, choline, inositol, betaine, and linoleic acid, fish oil, including components thereof such as omega-3 (N-3) polyunsaturated fatty acids, eicosapentaenoic acid and docosahexaenoic acid, coenzyme Q10, and/or combinations thereof. The oral care composition of the invention may comprise neutral amino acids, wherein the neutral amino acid is selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and/or combinations thereof; wherein preferably selected from glycine, asparagine, glutamine, and/or combinations thereof; and wherein the neutral amino acid is present in the amount of from 0.1% by weight to 5% by weight based on the total weight of the composition. The oral care composition of the invention may comprise anti-inflammatory agents. In some embodiments, the anti-inflammatory agents include steroidal agents such as fluocinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirine, diflunisal, meclofenamate, mefenamic acid, oxyphenbutazone and phenylbutazone, and/or combinations thereof.

In some embodiments, the oral care compositions disclosed herein may comprise an ingredient selected from: a humectant, an abrasive, a surfactant, a thickening agent, a foam modulator, a stain prevention agent, a preservative, an erosion prevention agent, an antiplaque agent, a sweetener, water, and combinations thereof.

In some embodiments, the oral care compositions disclosed herein may comprise at least one humectant from about 30.00% by weight to about 70.00% by weight, at least one abrasive from about 15.00% by weight to about 45.00% by weight, at least one surfactant from about 1.10% by weight to about 9.00% by weight, at least one thickening agent from about 0.20% by weight to about 8.00% by weight at least one foam modulator from about 1.00% by weight to about 6.00% by weight, at least one stain prevention agent from about 0.20% by weight to about 2.00% by weight, at least one preservative from about by weight to about 2.00% by weight, at least one erosion prevention agent from about by weight to about 10.00% by weight, at least one antiplaque agent from about 0.50% to about 5.00%, at least one sweetener from about 0.05% by weight to about 2.00% by weight, at least one whitening agent from about 0.01% by weight to about 6.00% by weight and/or combinations thereof, wherein the weights are based on the total weight of the composition.

In at least one embodiment, the oral care compositions disclosed herein comprises:
a natural sweetener system comprising 95%-99% by wt. % pure Rebaudioside A extract (where the wt. % is relative to the total weight of the extract), and enzymatically glucosylated steviol glycosides extract, wherein the enzymatically glucosylated steviol glycosides extract are 80% weight to 98% by wt. % pure (where the wt. % is relative to the total weight of the extract); wherein the natural sweetener system is in an amount from about 0.05% to about 3.00% weight, based on the total weight of the composition, and wherein the (wt %) ratio of Rebaudioside A extract to enzymatically glucosylated steviol glycosides extract is 1:1, or 1:2, or 1:3, or 1:4, or 1:5, or 2:1, or 3:1, or 4:1, or 5:1; and an orally acceptable carrier, wherein the orally acceptable carrier.

In at least one embodiment, the oral care compositions disclosed herein comprises:
a natural sweetener system comprising 95%-99% by wt. % pure Rebaudioside A extract (where the wt. % is relative to the total weight of the extract), and enzymatically glucosylated steviol glycosides extract, wherein the enzymatically glucosylated steviol glycosides extract are 80% weight to 98% by wt. % pure (where the wt. % is relative to the total weight of the extract); wherein the natural sweetener system is in an amount from about 0.05% to about 3.00% weight, based on the total weight of the composition, and wherein the (wt %) ratio of Rebaudioside A extract to enzymatically glucosylated steviol glycosides extract is 1:1, or 1:2, or 1:3, or 1:4, or 1:5, or 2:1, or 3:1, or 4:1, or 5:1; and an orally acceptable carrier, wherein the orally acceptable carrier comprises one or more ingredients selected from:
a humectant from about 30% by weight to about 70% by weight;
an abrasive from about 15% by weight to about 45% by weight;
a surfactant from about 1.1% by weight to about 9% by weight;
a thickening agent from about 0.2% by weight to about 8% by weight;
a foam modulator from about 1% by weight to about 6% by weight;
a stain prevention agent from about 0.2% by weight to about 2% by weight;
a preservative from about 0.2% by weight to about 2% by weight;
an erosion prevention agent from about 0.2% by weight to about 10% by weight;
an antiplaque agent from about 0.5% to about 5%;
a sweetener from about 0.05% by weight to about 2% by weight;
a whitening agent from about 0.01% by weight to about 6% by weight; and combinations thereof, wherein the weight % of the ingredients is based on the total weight of the oral care composition.

In at least one embodiment, the oral care compositions disclosed herein comprise at least one humectant from about 20% by weight to about 70% by weight, or from about 20% by weight to about 45% by weight, or from about 45% by weight to about 50% by weight, or from about 50% by weight to about 60% by weight, or from about 60% by weight to about 70% by weight, relative to the total weight of the oral care composition.

In at least one embodiment, the oral care compositions disclosed herein comprise at least one abrasive from about 5% by weight to about 45% by weight, or from about 5% by weight to about 10% by weight, or from about 10% by weight to about 20% by weight, or from about 20% by weight to about 35% by weight, or from about 35% by weight to about 45% by weight, relative to the total weight of the oral care composition.

In a further embodiment, the oral care compositions disclosed herein comprise at least one anionic surfactant from about 0.30% by weight to about 6.00% by weight, or from about 0.30% by weight to about 0.70% by weight, or from about 0.70% by weight to about 1% by weight, or from about 1% by weight to about 2% by weight, or from about 2% by weight to about 4% by weight, or from about 4% by weight to about 4.5% by weight, or from about 4.5% by weight to about 5% by weight, or from about 5% by weight to about 6% by weight, relative to the total weight of the oral care composition.

In a further embodiment, the oral care compositions disclosed herein comprise at least one nonionic surfactant from about 0.01% by weight to about 10% by weight, or from about 0.01% by weight to about 0.05% by weight, or from about 0.05% by weight to about 1% by weight, or from about 1% by weight to about 3% by weight, or from about 3% by weight to about 5% by weight, or from about 5% by weight to about 7% by weight, or from about 7% by weight to about 9% by weight, or from about 9% by weight to about 10% by weight, relative to the total weight of the oral care composition.

In a further embodiment, the oral care compositions disclosed herein comprise at least one amphoteric surfactant from about 0.08% by weight to about 3% by weight, or from about 0.80% by weight to about 1% by weight, or from about 1% by weight to about 1.60% by weight, or from about 1.60% by weight to about 2% by weight, or from about 2% by weight to about 2.6% by weight, or from about 2.6% by weight to about 3% by weight, relative to the total weight of the oral care composition.

In a further embodiment, the oral care compositions disclosed herein comprise at least one thickening agent from about 0.20% by weight to about 8% by weight, or from about 0.20% by weight to about 0.50% by weight, or from about 0.50% by weight to about 1.50% by weight, or from about 1.50% by weight to about 2.50% by weight, or from about 2.50% by weight to about 3.50% by weight, or from about 3.50% by weight to about 5.00% by weight, or from about 5.00% by weight to about 5.50% by weight, or from about 5.50% by weight to about 7.00% by weight, or from about 7.00% by weight to about 8.00% by weight, relative to the total weight of the oral care composition.

In a further embodiment, the oral care compositions disclosed herein comprise at least one foam modulator from about 1% by weight to about 6% by weight, or from about 1% by weight to about 2% by weight, or from about 2% by weight to about 3.5% by weight, or from about 3.5% by weight to about 5% by weight, or from about 5% by weight to about 6% by weight, relative to the total weight of the oral care composition.

In a further embodiment, the oral care compositions disclosed herein comprise at least one stain prevention agent from about 0.20% by weight to about 3% by weight, or from about 0.20% by weight to about 0.45% by weight, or from about 0.45% by weight to about 0.65% by weight, or from about 0.65% by weight to about 0.85% by weight, or from about 0.85% by weight to about 1% by weight, or from about 1% by weight to about 3% by weight, relative to the total oral care composition.

In a further embodiment, the oral care compositions disclosed herein comprise at least one preservative from about 0.20% by weight to about 1% by weight, or from about by weight to about 0.45% by weight, or from about 0.45% by weight to about 0.55% by weight, or from about 0.55% by weight to about 0.85% by weight, or from about 0.85% by weight to about 1% by weight, relative to the total oral care composition.

In a further embodiment, the oral care compositions disclosed herein comprise at least one erosion prevention agent from about 0.20% by weight to about 10% by weight, or from about 0.20% by weight to about 0.35% by weight, or from about 0.35% by weight to about 0.65% by weight, or from about 0.65% by weight to about 0.85% by weight, or from about 0.85% by weight to about 1.00% by weight, or from about 1% by weight to about 4% by weight, or from about 4.00% by weight to about 7% by weight, or from about 7% by weight to about 10% by weight, relative to the total oral care composition.

In a further embodiment, the oral care compositions disclosed herein comprise at least one antiplaque agent from about 0.50% by weight to about 45% by weight, or from about 0.50% by weight to about 1% by weight, or from about 1% by weight to about 2% by weight, or from about 2% by weight to about 3.2% by weight, or from about 3.2% by weight to about 4% by weight, or from about 4% by weight to about 5% by weight, or from about 5% by weight to about 25% by weight, or from about 25% by weight to about 35% by weight, or from about 35% by weight to about 40% by weight, or from about 40% by weight to about 45% by weight, relative to the total oral care composition.

In a further embodiment, the oral care compositions disclosed herein comprise at least one sweetener from about 0.05% by weight to about 3% by weight, or from about by weight to about 0.10% by weight, or from about 0.10% by weight to about 0.6% by weight, or from about 0.6% by weight to about 1% by weight, or from about 1% by weight to about 1.2% by weight, or from about 1.2% by weight to about 1.7% by weight, or from about 1.7% by weight to about 2% by weight, or from about 2% by weight to about 2.2% by weight, or from about 2.2% by weight to about 2.5% by weight, or from about 2.5% by weight to about 2.7% by weight, or from about 2.70% by weight to about 3.00% by weight, relative to the total weight of the oral care composition.

As used herein, “anhydrous”, refers to less than about 5% by weight of water, optionally less than about 4% by weight, less than about 3% by weight, less than about 2% by weight, less than about 1% by weight, less than about 0.5% by weight, less than about 0.1% by weight down to about 0% by weight water. In certain embodiments, the oral care composition comprises water in an amount from about 60% by weight to about 90% by weight based on the total weight of the composition. In certain embodiments, the oral care composition comprises water in an amount from about 65% by weight to about 85% by weight based on the total weight of the oral care composition. In certain embodiments, the oral care composition comprises water in an amount from about 70% by weight to about 80% by weight based on the total weight of the composition. In certain embodiments, the oral care composition comprises water in an amount of about 10% by weight to about 22% by weight of the total weight of the oral care composition.

It is understood that while general attributes of each of the above categories of materials may differ, there may be some common attributes and any given material may serve multiple purposes within two or more of such categories of materials.

In certain embodiments, the oral care compositions described herein are anhydrous. It is understood that toothpaste for example will typically be diluted with water upon use, while a mouth rinse typically will not be. The compositions of the invention may comprise various agents which are active to protect and enhance the strength and integrity of the enamel and tooth structure and/or to reduce bacteria and associated tooth decay and/or gum disease. Effective concentration of the active ingredients used herein will depend on the particular agent and the delivery system used.

Unless stated otherwise, all percentages of the oral care composition components given in this specification are by weight, based on a total composition or formulation weight of 100%.

In various embodiments, the natural sweetener system of the invention comprising a Reb A 95% to 99% by wt. pure extract (where the % wt. is relative to the total extract) and GSG extract wherein the GSG a 80% weight to 98% weight pure extract, (relative to the total weight of the extract), can be used in any orally consumable product, such as but not limited to: beverages, foods, confectionary, soft drinks, canned fruits and jams, ice cream, dairy products, cakes, deserts, alcoholic beverages. The disclosure provides in one embodiment any orally consumable product, such as but not limited to: beverages, foods, confectionary, soft drinks, canned fruits and jams, ice cream, dairy products, cakes, deserts, alcoholic beverages, comprising a natural sweetener system Reb A extract in a purity of from 95% to 99% by wt. (relative to the total weight of the extract) and GSG extract wherein the GSG is in 80% weight to 98% weight (relative to the extract), and the ratio of Reb A to GSG80 is 1:1, or 1:2, or 1:3, or, 1:4, or, 1:5, or 2:1, or 3:1, or 4:1, or 5:1, respectively.

Methods of Manufacture

The oral care compositions of the disclosure are made by any variety of methods including adding and mixing the ingredients of the composition in a suitable vessel provided with a mixer. In one embodiment, humectant(s), surfactant(s) and carrier(s) are mixed to form a homogenous mixture. Additional ingredients such as flavoring or coloring agents are added at any point during the mixing process but in various embodiments such ingredients are preferably added last or close to last. Depending on the product to be made and the amount of water within, the sweetener system may be premixed with the water, or the sweetener system may be added at the end of the process.

### EXAMPLES

The following examples are for illustrative purposes only and are not intended, nor should they be construed as limiting the invention in any manner Those skilled in the art will appreciate that variations and modifications of the following examples may be made without exceeding the spirit or scope of the invention. As will be evident to those skilled in the art, individual isomeric forms can be obtained by separation of mixtures thereof in conventional manner. For example, in the case of diasteroisomeric isomers, chromatographic separation may be employed.

Quality of sweetness performance of the natural sweetener in various toothpaste bases is ranked on a scale of 0 to 5 by a flavor expert. The tables below indicate relevance of the scores is indicated in Table A.

TABLE A
Sweetness scores

| | Sweetness | Comments |
|---|---|---|
| Score | 5 | Highest organoleptic performance of sweetness, |
| | | pleasant similar to sucrose, no lingering aftertaste |
| Score | 4 | Clean and pleasant sweetness that is full bodied, |
| | | but with slight lingering aftertaste |
| Score | 3 | Moderate sweetness with a lingering aftertaste |
| Score | 2 | Mild sweetness, unbalanced in sweetness onset with a |
| | | lingering aftertaste |
| Score | 1 | Minimal amount of sweetness |
| Score | 0 | No sweetness perceived |

Quality of bitterness performance is ranked on a scale of 0 to 5 by a flavor expert. The tables below indicate relevance of the scores is indicated in Table B.

TABLE B
Bitterness Scores

| | Bit-terness | Comments |
|---|---|---|
| Score | 5 | Extreme amount of bitterness, sharp, and harsh |
| | | taste that lingers |
| Score | 4 | High bitterness, unacceptable |
| Score | 3 | Moderate bitterness that is borderline acceptable |
| Score | 2 | Perceivable bitterness that is acceptable |
| Score | 1 | Slight bitterness that may not be detectable by |
| | | a non-expert |
| Score | 0 | No perceived bitterness |

### Example 1: Preparation of Basic Unsweetened Toothpastes

The following tables represent different backbones of toothpaste base formulations prior to the addition of the natural sweetener system. After the addition of the natural sweetener system of the invention the toothpaste formulations are tested for organoleptic properties. The quality of sweetness and bitterness performance of the natural sweetener in various toothpaste bases is ranked by a flavor expert on a scale of 0 to 5 by a flavor expert and the results are detailed in Example 2.

TABLE 2
Toothpaste A

| | | Weight |
|---|---|---|
| | Ingredients | (%) |
| 1 | Sorbitol (non-crystal, 70% solution) | 68 |
| 2 | Abrasive | 8 |
| | (e.g., high cleaning silica, potassium silicate, perlite, | |
| | synthetic amorphous silica, synthetic abrasive silica, core | |
| | shell silica, precipitated silica, sodium bicarbonate, | |
| | precipitated calcium carbonate, dicalcium phosphate) | |
| 3 | Sodium Lauryl Sulfate | 1.5 |
| 4 | Thickening Agent | 8 |
| | (e.g., thickening silica, xanthan gum, gums, carbomers, | |
| | carrageenans, sodium carboxymethyl cellulose, polyanionic | |
| | cellulose, fumed silica) | |
| 5 | Sodium carboxymethyl cellulose | 0.65 |
| 6 | Sodium Fluoride | 0.25 |
| 7 | Xylitol | 5 |
| 8 | Demineralized water | q.s. |
| 9 | Polyethylene glycol | 1 |
| 10 | Preservative | 0.5 |
| 11 | Flavor | 1 |

TABLE 3
Toothpaste B

| | | Weight |
|---|---|---|
| | Ingredients | (%) |
| 1 | Sorbitol (non-crystal, 70% solution) | 21 |
| 2 | Abrasive | 42 |
| | (e.g., high cleaning silica, potassium silicate, perlite, | |
| | synthetic amorphous silica, synthetic abrasive silica, core | |
| | shell silica, precipitated silica, sodium bicarbonate, | |
| | precipitated calcium carbonate, dicalcium phosphate) | |
| 3 | Anionic Surfactant | 2 |
| 4 | Thickening Silica | 4.8 |
| 5 | Sodium carboxymethyl cellulose | 1.1 |
| 6 | L- Arginine | 1.5 |
| 7 | Preservative | 0.3 |
| 8 | Demineralized Water | q.s. |
| 9 | pH adjusters | 0.85 |
| 10 | Flavor | 1.3 |

TABLE 4
Toothpaste C

| | | Weight |
|---|---|---|
| | Ingredients | (%) |
| 1 | Sorbitol - Non-Crystal - 70% Solution | 59 |
| 2 | Glycerin | 3 |
| 3 | Abrasives | 20 |
| | (e.g. high cleaning silica, potassium silicate, perlite, | |
| | synthetic amorphous silica, synthetic abrasive silica, core | |
| | shell silica, precipitated silica, sodium bicarbonate, | |
| | precipitated calcium carbonate, dicalcium phosphate) | |
| 4 | Anionic Surfactants | 5 |
| | (e.g. cocoyl monoglyceride sulfonate, sodium lauryl | |
| | sarcosinate, sodium lauryl isoethionate, sodium laureth | |
| | carboxylate, sodium lauryl sulfate, sodium cocoyl | |
| | glutamate, sulfonated monoglycerides of C₈₋₂₀ fatty acids, | |
| | sodium stearate, lauryl glucoside) | |
| 5 | Amphoteric surfactants | 0.50 |
| | (e.g. cocamidopropyl betaine, alkyl betaines and alkyl | |
| | amido betaines) | |
| 6 | Thickening Agents | 5 |
| | (e.g. thickening silica, xanthan gum, gums, carbomers, | |
| | carrageenans, sodium carboxymethyl cellulose, polyanionic | |
| | cellulose, fumed silica) | |
| 7 | Sodium fluoride | 0.24 |
| 8 | Demineralized water | q.s. |
| 9 | Flavor | 1.2 |

TABLE 5
Toothpaste D

| | | Weight |
|---|---|---|
| | Ingredients | (%) |
| 1 | Polyethylene Glycol/Polypropylene Glycol Copolymer | 7.5 |
| 2 | Block copolymer of ethylene oxide and | 47 |
| | propylene oxide | |
| 2 | Calcium pyrophosphate | 15 |
| 3 | Thickening Agents | 19.5 |
| 4 | Sodium lauryl sulfate powder | 2 |
| 5 | Tetrasodium pyrophosphate | 1.30 |
| 7 | Sodium monofluorophosphate | 0.80 |
| 8 | Hydrogen peroxide | 4.00 |
| 9 | Sodium acid pyrophosphate | 0.4 |
| 10 | Flavor and Antioxidant agent | 2 |

TABLE 6
Toothpaste E

| | | Weight |
|---|---|---|
| | Ingredients | (%) |
| 1 | Sorbitol - Non-Crystal - 70% Solution | 31 |
| 2 | Anionic Surfactants | 2 |
| | (e.g. sodium carboxymethyl cellulose, polyanionic | |
| | cellulose, cocoyl monoglyceride sulfonate, sodium lauryl | |
| | sarcosinate, sodium lauryl isoethionate, sodium laureth | |
| | carboxylate, sodium lauryl sulfate, sodium cocoyl | |
| | glutamate, sulfonated monoglycerides of C₈₋₂₀ fatty | |
| | acids, sodium stearate, lauryl glucoside) | |
| 3 | Thickening Agent | 1 |
| | (e.g. thickening silica, xanthan gum, gums, carbomers, | |
| | carrageenans, sodium carboxymethyl cellulose, polyanionic | |
| | cellulose, fumed silica) | |
| 4 | Tetrasodium pyrophosphate | 1.1 |
| 5 | Dicalcium phosphate dihydrate | 45 |
| 6 | Sodium monofluorophosphate | 1.1 |
| 7 | Demineralized water | q.s. |
| 8 | Flavor | 3.2 |

TABLE 7
Toothpaste F

| | | Weight |
|---|---|---|
| | Ingredients | (%) |
| 1 | 99.5% vegetable refined glycerin | 35 |
| 2 | Abrasives | 15 |
| | (e.g. high cleaning silica, potassium silicate, perlite, | |
| | synthetic amorphous silica, synthetic abrasive silica, core | |
| | shell silica, precipitated silica, sodium bicarbonate, | |
| | dicalcium phosphate, precipitated calcium carbonate) | |
| 3 | Anionic Surfactant | 2.5 |
| | (e.g. sodium carboxymethyl cellulose, polyanionic | |
| | cellulose, cocoyl monoglyceride sulfonate, sodium lauryl | |
| | sarcosinate, sodium lauryl isoethionate, sodium laureth | |
| | carboxylate, sodium lauryl sulfate, sodium cocoyl | |
| | glutamate, sulfonated monoglycerides of C₈₋₂₀ fatty acids, | |
| | sodium stearate, lauryl glucoside, sodium methyl | |
| | cocoyl taurate) | |
| 4 | Amphoteric surfactant | 3 |
| | (e.g. cocamidopropyl betaine, alkyl betaines and alkyl | |
| | amido betaines) | |
| 5 | Thickening Agents | 7.4 |
| | (e.g. thickening silica, xanthan gum, gums, carbomers, | |
| | carrageenans, sodium carboxymethyl cellulose, polyanionic | |
| | cellulose, fumed silica) | |
| 6 | Stain Prevention Agents | 0.50 |
| | (e.g. tetrasodium pyrophosphate, disodium pyrophosphate, | |
| | sodium tripyrophosphate, sodium hexametaphosphate, | |
| | zinc citrate) | |
| 7 | pH adjuster | 0.35 |
| 8 | L-Arginine | 1.5 |
| 9 | Sodium Fluoride | 0.32 |
| 10 | Tetrasodium pyrophosphate | 0.5 |
| 11 | Zinc citrate | 0.5 |
| 12 | Zinc oxide | 1 |
| 13 | Demineralized water | q.s. |
| 14 | Flavor and color | 2.35 |

TABLE 8
Toothpaste G

| | | Weight |
|---|---|---|
| | Ingredients | (%) |
| 1 | Glycerin | 35 |
| 2 | Precipitated calcium carbonate | 37 |
| 3 | Anionic Surfactants | 2.5 |
| | (e.g. sodium carboxymethyl cellulose, polyanionic | |
| | cellulose, cocoyl monoglyceride sulfonate, sodium lauryl | |
| | sarcosinate, sodium lauryl isoethionate, sodium laureth | |
| | carboxylate, sodium lauryl sulfate, sodium cocoyl | |
| | glutamate, sulfonated monoglycerides of C₈₋₂₀ fatty acids, | |
| | sodium stearate, lauryl glucoside) | |
| 4 | Amphoteric surfactants | 3 |
| | (e.g. cocamidopropyl betaine, alkyl betaines and alkyl | |
| | amido betaines) | |
| 5 | Carboxymethyl cellulose | 1 |
| 6 | Anticalculus /Antitartar Agents | 1.1 |
| | (e.g. stannous ion sources, polycarboxylate | |
| | polymers, polyaminopropanesulfonic acid, azacycloalkane- | |
| | 2,2-diphosphonates, tetrasodium pyrophosphates, calcium | |
| | pyrophosphate, alumina, dicalcium orthophosphate | |
| | dihydrate, n-calcium pyrophosphate, tricalcium phosphate, | |
| | calcium polymetaphosphate, insoluble sodium | |
| | polymetaphosphate) | |
| 7 | Sodium monofluorophosphate | 1.1 |
| 8 | Demineralized water | q.s. |
| 9 | pH adjuster | 0.5 |
| 10 | Flavor and color | 3.3 |

TABLE 9
Toothpaste H

| | | Weight |
|---|---|---|
| | Ingredients | (%) |
| 1 | Non-crystal sorbitol, 70% solution | 50 |
| 2 | Glycerin | 2 |
| 3 | Abrasives | 15 |
| | (e.g. high cleaning silica, potassium silicate, perlite, synthetic | |
| | amorphous silica, synthetic abrasive silica, core shell silica, | |
| | precipitated silica, precipitated calcium carbonate, | |
| | dicalcium phosphate, sodium bicarbonate,) | |
| 4 | 35% Sodium lauryl sulfate - Liquid | 5.7 |
| 5 | Amphoteric surfactants | 1.25 |
| | (e.g. cocamidopropyl betaine, alkyl betaines and alkyl amido betaines) | |
| 6 | Thickening Agents | 6.3 |
| | (e.g. thickening silica, xanthan gum, gums, carbomers, carrageenans, | |
| | sodium carboxymethyl cellulose, polyanionic cellulose, fumed silica) | |
| 7 | Tetrasodium pyrophosphate | 0.5 |
| 8 | Sodium fluoride | 0.32 |
| 9 | Zinc citrate trihydrate | 0.5 |
| 10 | Zinc oxide | 1.0 |
| 11 | Demineralized water | q.s. |
| 12 | Preservative | 0.3 |

### Example 3: Evaluation of the Natural Sweetener System of the Invention in Combination with Xylitol

A commonly used natural ingredient in oral care is xylitol. It is frequently incorporated into toothpaste formulas for its humectant properties, perceived anticariogenic benefits, and for providing sweetness to toothpaste. Given that xylitol provides multiple functions in a toothpaste formula, it cannot be removed from certain products without necessitating a large formula change. However, xylitol may not provide the sweetness intensity needed to cover some negative taste attributes from the base. Therefore, the new sweetener system comprising 98% pure Reb A and GSG80 in different ratios, is added to Toothpaste A which already comprises 5% by weight of xylitol. Toothpaste A samples are evaluated for their organoleptic properties; the results are tabulated in Table 10. The wt % is by wt % of the total toothpaste sample.

TABLE 10
Organoleptic Results for the Natural Sweetener System in Combination
with Xylitol formulated with Toothpaste Base A^{a}

| | Materials and Amounts Used | | | Ratio | | |
|---|---|---|---|---|---|---|
| | in Toothpaste Samples (%) | | | Reb A^{b} to | | |
| Sample | Xylitol | Reb A^{a} | GSG80 | GSG80 | Sweetness ^{c} | Bitterness ^{d} |
| 1 | 5 | 0 | 0 | — | 1 | 4-5 |
| 2 | 5 | 0 | 0.10 | 0:1 | 2 | 4 |
| 3 | 5 | 0.05 | 0.05 | 1:1 | 3 | 4 |
| 4 | 5 | 0.033 | 0.067 | 1:2 | 4 | 2 |
| 5 | 5 | 0.025 | 0.075 | 1:3 | 4 | 3 |
| 6 | 5 | 0.02 | 0.08 | 1:4 | 3 | 4 |
| 7 | 5 | 0.017 | 0.083 | 1:5 | 3 | 4 |
| 8 | 5 | 0.067 | 0.033 | 2:1 | 4 | 4 |
| 9 | 5 | 0.075 | 0.025 | 3:1 | 4 | 3 |
| 10 | 5 | 0.08 | 0.02 | 4:1 | 4 | 3 |
| 11 | 5 | 0.083 | 0.017 | 5:1 | 4 | 3 |

^{a}Toothpaste Base A according to Table 2
^{b}Reb A is 98% pure, e.g., 98% Reb A relative to the weight of the Reb A extract
^{c} Sweetness scale of 0-5 according to Table A
^{d} Bitterness scale of 0-5 according to Table B

Using 98% pure Reb A with GSG80 in a 1:2 ratio provide a significant benefit to the taste profile of Toothpaste A containing only xylitol. It is observed that using higher ratios of 98% pure Reb A results in similar sweetness quality but the bitterness increased in intensity and that these higher ratios of 98% Reb A decrease the perceived flavor intensity. Overall, the natural sweetener system comprising 98% Reb A and GSG80 in a 1:2 ratio, in the presence of 5% xylitol, provides a significant benefit by masking off-tastes in natural toothpaste bases and provided a clean sweetness profile.

### Example 4: Evaluation of the Natural Sweetener System of the Invention, as the Sole Sweetener in Different Toothpaste Formulations

The natural sweetener system of the invention is evaluated in the unsweetened toothpastes defined in the tables of Example 2. Usage levels and organoleptic results are listed in the following tables, for each toothpaste described in the tables of Example 2.

TABLE 11
Organoleptic Results for the Natural Sweetener
System in Toothpaste Base B^{a}

| | Amount of | | | |
|---|---|---|---|---|
| | Combination | | | |
| | Reb A^{b} and GSG80 | Ratio of | | |
| | Used in Toothpaste | Reb A^{b} | | |
| Sample | Samples (%) | to GSG80 | Sweetness ^{c} | Bitterness ^{d} |
| 1 | 0.90 | 0:1 | 3 | 3 |
| 2 | 0.90 | 1:1 | 4 | 2 |
| 3 | 0.90 | 1:2 | 5 | 1 |
| 4 | 0.90 | 1:3 | 4 | 3 |
| 5 | 0.90 | 1:4 | 4 | 3 |
| 6 | 0.90 | 1:5 | 3 | 3 |
| 7 | 0.90 | 2:1 | 4 | 2 |
| 8 | 0.90 | 3:1 | 4 | 2 |
| 9 | 0.90 | 4:1 | 5 | 2 |
| 10 | 0.90 | 5:1 | 5 | 1 |

^{a}Toothpaste B according to Table 3
^{b}Reb A is 98% pure, e.g., 98% Reb A relative to the weight of the Reb A extract
^{c} Sweetness scale of 0-5 according to Table A
^{d} Bitterness scale of 0-5 according to Table B

TABLE 12
Organoleptic Results for the Natural Sweetener
System in Toothpaste Base C^{a}

| | Amount of | | | |
|---|---|---|---|---|
| | Combination | | | |
| | Reb A^{b} and GSG80 | Ratio of | | |
| | Used in Toothpaste | Reb A^{b} | | |
| Sample | Samples (%) | to GSG80 | Sweetness ^{c} | Bitterness ^{d} |
| 1 | 0.90 | 0:1 | 3 | 4 |
| 2 | 0.90 | 1:1 | 4 | 3 |
| 3 | 0.90 | 1:2 | 5 | 1 |
| 4 | 0.90 | 1:3 | 4 | 3 |
| 5 | 0.90 | 1:4 | 3 | 3 |
| 6 | 0.90 | 1:5 | 3 | 3 |
| 7 | 0.90 | 2:1 | 4 | 2 |
| 8 | 0.90 | 3:1 | 5 | 2 |
| 9 | 0.90 | 4:1 | 5 | 2 |
| 10 | 0.90 | 5:1 | 5 | 2 |

^{a}Toothpaste C according to Table 4
^{b}Reb A is 98% pure, e.g., 98% Reb A relative to the weight of the Reb A extract
^{c} Sweetness scale of 0-5 according to Table A
^{d} Bitterness scale of 0-5 according to Table B

TABLE 13
Organoleptic Results for the Natural Sweetener
System in Toothpaste Base D^{a}

| | Amount of | | | |
|---|---|---|---|---|
| | Combination | | | |
| | Reb A^{b} and GSG80 | Ratio of | | |
| | Used in Toothpaste | Reb A^{b} | | |
| Sample | Samples (%) | to GSG80 | Sweetness ^{c} | Bitterness ^{d} |
| 1 | 0.90 | 0:1 | 1 | 4 |
| 2 | 0.90 | 1:1 | 2 | 4 |
| 3 | 0.90 | 1:2 | 3 | 3 |
| 4 | 0.90 | 1:3 | 2 | 3 |
| 5 | 0.90 | 1:4 | 2 | 4 |
| 6 | 0.90 | 1:5 | 2 | 4 |
| 7 | 0.90 | 2:1 | 3 | 3 |
| 8 | 0.90 | 3:1 | 3 | 3 |
| 9 | 0.90 | 4:1 | 4 | 3 |
| 10 | 0.90 | 5:1 | 4 | 3 |

^{a}Toothpaste D according to Table 5
^{b}Reb A is 98% pure, e.g., 98% Reb A relative to the weight of the Reb A extract
^{c} Sweetness scale of 0-5 according to Table A
^{d} Bitterness scale of 0-5 according to Table B

TABLE 14
Organoleptic Results for the Natural
Sweetener System in Toothpaste E^{a}

| | Amount of | | | |
|---|---|---|---|---|
| | Combination | | | |
| | Reb A^{b} and GSG80 | Ratio of | | |
| | Used in Toothpaste | Reb A^{b} | | |
| Sample | Samples (%) | to GSG80 | Sweetness ^{c} | Bitterness ^{d} |
| 1 | 0.90 | 0:1 | 2 | 4 |
| 2 | 0.90 | 1:1 | 3 | 3 |
| 3 | 0.90 | 1:2 | 4 | 3 |
| 4 | 0.90 | 1:3 | 3 | 4 |
| 5 | 0.90 | 1:4 | 3 | 4 |
| 6 | 0.90 | 1:5 | 3 | 4 |
| 7 | 0.90 | 2:1 | 4 | 3 |
| 8 | 0.90 | 3:1 | 4 | 3 |
| 9 | 0.90 | 4:1 | 4 | 2 |
| 10 | 0.90 | 5:1 | 4 | 2 |

^{a}Toothpaste E according to Table 6
^{b}Reb A is 98% pure, e.g., 98% Reb A relative to the weight of the Reb A extract
^{c} Sweetness scale of 0-5 according to Table A
^{d} Bitterness scale of 0-5 according to Table B

TABLE 15
Organoleptic Results for the Natural Sweetener System in Toothpaste Base F^{a}

| | | | Amounts | | Ratio of | | |
|---|---|---|---|---|---|---|---|
| | | | Used in | | Reb A^{b} | | |
| | | | Toothpaste | | to | | |
| Sample | Materials | | Samples (%) | | GSG80 | Sweetness ^{c} | Bitterness ^{d} |
| I | Saccharin | | 0.3 | | — | 4 | 3 |
| II | Saccharin * | Sucralose ** | 0.3* | 0.02** | — | 4 | 3 |

| | | | | | | |
|---|---|---|---|---|---|---|
| 1 | Reb A^{a} | GSG80 | 0.90 | 0:1 | 2 | 4 |
| 2 | Reb A^{a} | GSG80 | 0.90 | 1:1 | 3 | 3 |
| 3 | Reb A^{a} | GSG80 | 0.90 | 1:2 | 4 | 3 |
| 4 | Reb A^{a} | GSG80 | 0.90 | 1:3 | 4 | 4 |
| 5 | Reb A^{a} | GSG80 | 0.90 | 1:4 | 3 | 4 |
| 6 | Reb A^{a} | GSG80 | 0.90 | 1:5 | 3 | 4 |
| 7 | Reb A^{a} | GSG80 | 0.90 | 2:1 | 4 | 3 |
| 8 | Reb A^{a} | GSG80 | 0.90 | 3:1 | 4 | 3 |
| 9 | Reb A^{a} | GSG80 | 0.90 | 4:1 | 4 | 2 |
| 10 | Reb A^{a} | GSG80 | 0.90 | 5:1 | 4 | 2 |

^{a}Toothpaste F according to Table 7
^{b}Reb A is 98% pure, e.g., 98% Reb A relative to the weight of the Reb A extract
^{c} Sweetness scale of 0-5 according to Table A
^{d} Bitterness scale of 0-5 according to Table B

TABLE 16
Organoleptic Results for the Natural Sweetener
System in Toothpaste Base G^{a}

| | Amount of | | | |
|---|---|---|---|---|
| | Combination | | | |
| | Reb A^{b} and GSG80 | Ratio of | | |
| | Used in Toothpaste | Reb A^{b} | | |
| Sample | Samples (%) | to GSG80 | Sweetness ^{c} | Bitterness ^{d} |
| 1 | 0.60 | 0:1 | 3 | 3 |
| 2 | 0.60 | 1:1 | 4 | 3 |
| 3 | 0.60 | 1:2 | 5 | 1 |
| 4 | 0.60 | 1:3 | 4 | 3 |
| 5 | 0.60 | 1:4 | 4 | 3 |
| 6 | 060 | 1:5 | 3 | 3 |
| 7 | 0.60 | 2:1 | 4 | 3 |
| 8 | 0.60 | 3:1 | 5 | 3 |
| 9 | 0.60 | 4:1 | 5 | 2 |
| 10 | 0.60 | 5:1 | 5 | 2 |

^{a}Toothpaste G according to Table 8
^{b}Reb A is 98% pure, e.g., 98% Reb A relative to the weight of the Reb A extract
^{c} Sweetness scale of 0-5 according to Table A
^{d} Bitterness scale of 0-5 according to Table B

TABLE 17
Organoleptic Results for the Natural
Sweetener System in Toothpaste H^{a}

| | Amount of | | | |
|---|---|---|---|---|
| | Combination | | | |
| | Reb A^{b} and GSG80 | Ratio of | | |
| | Used in Toothpaste | Reb A^{b} | | |
| Sample | Samples (%) | to GSG80 | Sweetness ^{c} | Bitterness ^{d} |
| 1 | 0.90 | 0:1 | 3 | 4 |
| 2 | 0.90 | 1:1 | 4 | 3 |
| 3 | 0.90 | 1:2 | 5 | 1 |
| 4 | 0.90 | 1:3 | 4 | 3 |
| 5 | 0.90 | 1:4 | 3 | 3 |
| 6 | 0.90 | 1:5 | 3 | 3 |
| 7 | 0.90 | 2:1 | 4 | 2 |
| 8 | 0.90 | 3:1 | 5 | 2 |
| 9 | 0.90 | 4:1 | 5 | 2 |
| 10 | 0.90 | 5:1 | 5 | 2 |

^{a}Toothpaste H according to Table 9
^{b}Reb A is 98% pure, e.g., 98% Reb A relative to the weight of the Reb A extract
^{c} Sweetness scale of 0-5 according to Table A
^{d} Bitterness scale of 0-5 according to Table B

The tables hereinafter represent other possible backbones of toothpaste formulations, to which the natural sweetener system of the invention is added. The flavoring agents can be selected from a variety of fruity flavors:

TABLE 18
Toothpaste I

| | | Weight |
|---|---|---|
| | Ingredients | (%) |
| 1 | Humectants | 69 |
| | (e.g. non-crystal sorbitol, polyoxyethylene glycol 600) | |
| 2 | Abrasives | 8 |
| | (e.g. synthetic abrasive silica) | |
| 3 | Anionic Surfactants | 0.65 |
| | (e.g. sodium carboxymethyl cellulose, cocoyl monoglyceride sulfonate, | |
| | sodium lauryl sarcosinate) | |
| 4 | Thickening Agents | 8 |
| | (e.g. thickening silica, fumed silica) | |
| 5 | Foam Modulator | 1.5 |
| | (e.g. sodium lauryl sulfate powder, sodium lauryl sulfate liquid) | |
| 6 | Anticariogenic Agents | 5 |
| | (e.g. xylitol) | |
| 7 | Erosion prevention agents | 0.25 |
| | (e.g. Sodium fluoride) | |
| 8 | Preservatives | 0.5 |
| | (e.g. natural benzyl alcohol) | |
| 9 | Water, Flavoring agents | qs |

TABLE 19
Toothpaste J

| | | Weight |
|---|---|---|
| | Ingredients | (%) |
| 1 | Humectants | 23 |
| | (e.g. non-crystal sorbitol, polyoxyethylene glycol 600) | |
| 2 | Abrasives | 42.1 |
| | (e.g. sodium bicarbonate, calcium carbonate) | |
| 3 | Anionic Surfactants | 1.1 |
| | (e.g. sodium carboxymethyl cellulose, cocoyl monoglyceride sulfonate, | |
| | sodium lauryl sarcosinate) | |
| 4 | Thickening Agents | 2.4 |
| | (e.g. thickening silica, sodium carboxymethyl cellulose, polyanionic | |
| | cellulose, fumed silica) | |
| 5 | Foam Modulator | 5.7 |
| | (e.g. sodium lauryl sulfate liquid, sodium lauroyl sarcosinate) | |
| 6 | Erosion prevention agents | 1.5 |
| | (e.g. L-arginine, arginine phosphate, arginine hydrochloride, arginine | |
| | bicarbonate) | |
| 7 | Preservatives | 0.1 |
| | (e.g. natural benzyl alcohol) | |
| 8 | pH modifying agents | 0.75 |
| | (e.g. sodium carbonate, 85% syrupy phosphoric acid) | |
| 9 | Water, Flavoring agents | qs |

TABLE 20
Toothpaste K

| | | Weight |
|---|---|---|
| | Ingredients | (%) |
| 1 | Humectants | 30 |
| | (e.g. non-crystal sorbitol, 99.5% vegetable refined glycerin, polyhydric | |
| | alcohols, polyoxyethylene glycols) | |
| 2 | Abrasives | 42.1 |
| | (e.g. sodium bicarbonate, calcium carbonate) | |
| 3 | Anionic Surfactants | 0.9 |
| | (e.g. sodium carboxymethyl cellulose, cocoyl monoglyceride sulfonate, | |
| | sodium lauryl sarcosinate) | |
| 4 | Thickening Agents | 2.1 |
| | (e.g. thickening silica, sodium carboxymethyl cellulose, polyanionic | |
| | cellulose, fumed silica) | |
| 5 | Foam Modulator | 5.71 |
| | (e.g. 35% sodium lauryl sulfate liquid, sodium lauroyl sarcosinate) | |
| 6 | Erosion prevention agents | 1.5 |
| | (e.g. L-arginine, arginine phosphate, arginine hydrochloride, arginine | |
| | bicarbonate) | |
| 7 | pH modifying agents | 0.95 |
| | (e.g. sodium carbonate, 85% syrupy phosphoric acid) | |
| 8 | Water, Flavoring agents | qs |

TABLE 21
Toothpaste L

| | | Weight |
|---|---|---|
| | Ingredients | (%) |
| 1 | Humectants | 30 |
| | (e.g. non-crystal sorbitol, 99.5% vegetable refined glycerin, polyhydric | |
| | alcohols, polyoxyethylene glycols) | |
| 2 | Abrasives | 42.1 |
| | (e.g. sodium bicarbonate, calcium carbonate) | |
| 3 | Anionic Surfactants | 0.9 |
| | (e.g. sodium carboxymethyl cellulose, cocoyl monoglyceride sulfonate, | |
| | sodium lauryl sarcosinate) | |
| 4 | Thickening Agents | 2.1 |
| | (e.g. thickening silica, sodium carboxymethyl cellulose, polyanionic | |
| | cellulose, fumed silica) | |
| 5 | Foam Modulator | 5.71 |
| | (e.g. 35% sodium lauryl sulfate liquid, sodium lauroyl sarcosinate) | |
| 6 | Erosion prevention agents | 1.5 |
| | (e.g. L-arginine, arginine phosphate, arginine hydrochloride, arginine | |
| | bicarbonate) | |
| 7 | Preservatives | 0.1 |
| | (e.g. natural benzyl alcohol) | |
| 8 | pH modifying agents | 0.95 |
| | (e.g. sodium carbonate, 85% syrupy phosphoric acid) | |
| 9 | Water, Flavoring agents | qs |

TABLE 22
Toothpaste M

| | | Weight |
|---|---|---|
| | Ingredients | (%) |
| 1 | Humectants | 20 |
| | (e.g. vegetable refined glycerin) | |
| 2 | Abrasives | 42.5 |
| | (e.g. sodium bicarbonate, calcium carbonate) | |
| 3 | Anionic Surfactants | 1.2 |
| | (e.g. sodium carboxymethyl cellulose) | |
| 5 | Foam Modulator | 3.26 |
| | (e.g. sodium lauryl sulfate liquid, sodium lauryl sulfate powder) | |
| 6 | Erosion prevention agents | 1.5 |
| | (e.g. L-arginine, arginine phosphate, arginine hydrochloride, arginine | |
| | bicarbonate) | |
| 7 | Preservatives | 0.75 |
| | (e.g. natural benzyl alcohol) | |
| 8 | pH modifying agents | 0.35 |
| | (e.g. 85% syrupy phosphoric acid) | |
| 9 | Stain prevention agents | 0.75 |
| | (e.g. tetrasodium pyrophosphate, disodium pyrophosphate, sodium | |
| | tripyrophosphate) | |
| 10 | Water, Flavoring agents | qs |

TABLE 23
Toothpaste N

| | | Weight |
|---|---|---|
| | Ingredients | (%) |
| 1 | Humectants | 20 |
| | (e.g. vegetable refined glycerin) | |
| 2 | Abrasives | 42.5 |
| | (e.g. sodium bicarbonate, calcium carbonate) | |
| 3 | Anionic Surfactants | 0.8 |
| | (e.g. sodium carboxymethyl cellulose) | |
| 5 | Foam Modulator | 3.26 |
| | (e.g. sodium lauryl sulfate liquid, sodium lauryl sulfate powder) | |
| 6 | Erosion prevention agents | 2.26 |
| | (e.g. L-arginine, arginine phosphate, arginine hydrochloride, arginine | |
| | bicarbonate, sodium monofluorophosphate) | |
| 7 | Stain prevention agents | 0.75 |
| | (e.g. tetrasodium pyrophosphate, disodium pyrophosphate, sodium | |
| | tripyrophosphate) | |
| 8 | Preservatives | 0.75 |
| | (e.g. natural benzyl alcohol) | |
| 9 | pH modifying agents | 0.35 |
| | (e.g. 85% syrupy phosphoric acid) | |
| 10 | Water, Flavoring agents | qs |

TABLE 24
Toothpaste O

| | | Weight |
|---|---|---|
| | Ingredients | (%) |
| 1 | Humectants | 22 |
| | (e.g. vegetable refined glycerin) | |
| 2 | Abrasives | 42.5 |
| | (e.g. sodium bicarbonate, calcium carbonate) | |
| 3 | Anionic Surfactants | 1.2 |
| | (e.g. sodium carboxymethyl cellulose) | |
| 5 | Foam Modulator | 3.26 |
| | (e.g. sodium lauryl sulfate liquid, sodium lauryl sulfate powder) | |
| 6 | Erosion prevention agents | 1.5 |
| | (e.g. L-arginine, arginine, arginine phosphate, arginine hydrochloride, | |
| | arginine bicarbonate) | |
| 7 | Stain prevention agents | 0.75 |
| | (e.g. tetrasodium pyrophosphate, disodium pyrophosphate, sodium | |
| | tripyrophosphate) | |
| 8 | Preservatives | 0.75 |
| | (e.g. natural benzyl alcohol) | |
| 9 | pH modifying agents | 0.35 |
| | (e.g. 85% syrupy phosphoric acid) | |
| 10 | Water, Flavoring agents | qs |

TABLE 25
Toothpaste P

| | | Weight |
|---|---|---|
| | Ingredients | (%) |
| 1 | Humectants | 50 |
| | (e.g. non-crystal sorbitol, 99.5% vegetable refined glycerin polyhydric | |
| | alcohols, polyoxyethylene glycols) | |
| 2 | Abrasives | 5 |
| | (e.g. high cleaning silica, potassium silicate, perlite, synthetic | |
| | amorphous silica, synthetic abrasive silica, core shell silica, | |
| | precipitated silica, sodium bicarbonate, dicalcium phosphate, | |
| | precipitated calcium carbonate) | |
| 3 | Anionic Surfactants | 1.49 |
| | (e.g. sodium carboxymethyl cellulose, polyanionic cellulose, cocoyl | |
| | monoglyceride sulfonate, sodium lauryl sarcosinate, sodium lauryl | |
| | isoethionate, sodium laureth carboxylate, sodium lauryl sulfate, sodium | |
| | cocoyl glutamate, sulfonated monoglycerides of C₈₋₂₀ fatty acids, | |
| | sodium stearate, lauryl glucoside, sodium methyl cocoyl taurate) | |
| 4 | Thickening Agents | 6 |
| | (e.g. thickening silica, xanthan gum, gums, fumed silica) | |
| 5 | Stain Prevention Agents | 1 |
| | (e.g. tetrasodium pyrophosphate, disodium pyrophosphate, sodium | |
| | tripyrophosphate) | |
| 6 | Erosion prevention agents | 5 |
| | (e.g. L-arginine, arginine phosphate, arginine hydrochloride, arginine | |
| | bicarbonate,) | |
| 7 | Nutrients, Colorants, Flavoring agents, Fluoride salts, Saliva | qs |
| | stimulating source, Desensitizing agents, Preservatives, Antioxidants, | |
| | Water, pH Modifiers | |

What is claimed is:

1. An oral care composition comprising a natural sweetener system, wherein the natural sweetener system comprises:
an enzymatically glucosylated steviol glycoside extract; and
a rebaudioside A extract.

2. The oral care composition of claim 1, wherein the weight ratio of rebaudioside A extract to enzymatically glucosylated steviol glycosides extract is from 1:4 or to 5:1.

3. The oral care composition of claim 1, wherein the natural sweetener system is present in about 0.05 to about 3 wt. %, based on the total weight of the oral care composition.

4. The oral care composition of claim 1, wherein the rebaudioside A extract is present in an amount from 0.1 to 1 wt. %, based on the total weight of the oral care composition.

5. (canceled)

6. The oral care composition of claim 1, wherein the glucosylated steviol glycoside extract is present in an amount from 0.1 to 1 wt. %, based on the total weight of the oral care composition.

7. The oral care composition claim 1, wherein the enzymatically glucosylated steviol glycosides extract is comprised of 80 to 95 wt. % of glucosylated steviol glycosides by weight of the glucosylated steviol glycosides extract with the remainder being steviosides and rebaudioside that do not react with the enzyme.

8. The oral care composition of claim 1, wherein the oral care composition comprises from about 0.05 to about 10 wt. % of xylitol.

9. The oral care composition of claim 1, wherein the oral care composition comprises a stannous ion source selected from the group consisting of: stannous fluoride, stannous chloride, stannous pyrophosphate, stannous formate, stannous acetate, stannous gluconate, stannous lactate, stannous tartrate, stannous oxalate, stannous malonate, stannous citrate, stannous ethylene glyoxide, and combinations thereof.

10. The oral care composition of claim 9, wherein the stannous ion source comprises stannous fluoride.

11. The oral care composition of claim 1 further comprising a zinc ion source, wherein the zinc ion source comprises one or more zinc salt(s) selected from the group consisting of: zinc citrate, zinc oxide, zinc phosphate, zinc lactate, zinc sulfate, zinc silicate, zinc gluconate, and a combination of two or more thereof.

12. The oral care composition according to claim 11, wherein the oral care composition comprises a zinc ion source, wherein the zinc ion source comprises zinc oxide.

13. The oral care composition of claim 1 further comprising a zinc ion source, wherein the zinc ion source comprises zinc citrate.

14. The oral care composition according to claim 11, wherein zinc ion source comprises zinc phosphate.

15. The oral care composition of claim 1 further comprising an amino acid.

16. The oral care composition of claim 15, wherein the amino acid comprises arginine in an amount from 0.1 to 6.0 wt. %, based on the total weight of the oral care composition, wherein the weight of the arginine is calculated as free form.

17. The oral care composition of claim 1, wherein the composition comprises:
zinc phosphate; and
s-tannous fluoride.

18. The oral care composition according to claim 1, wherein the composition further comprises:
Zinc phosphate;
Stannous fluoride; and
Stannous pyrophosphate;
wherein the natural sweetener system further comprises xylitol.

19. The oral care composition according to claim 1, wherein the composition comprises:
zinc phosphate;
stannous fluoride; and
arginine;
wherein the rebaudioside A and the enzymatically glucosylated steviol glycosides extract and present in a weight ratio of rebaudioside A extract to enzymatically glucosylated steviol glycosides extract that is from 1:4 to 5:1.

20. The oral care composition according to claim 1, wherein the composition comprises:
zinc citrate; and
zinc oxide,
wherein the rebaudioside A and the enzymatically glucosylated steviol glycosides extract and present in a weight ratio of rebaudioside A extract to enzymatically glucosylated steviol glycosides extract that is from 1:4 to 5:1.

21. The oral care composition according to claim 1, wherein the composition comprises:
zinc citrate;
zinc oxide; and
arginine,
wherein the rebaudioside A and the enzymatically glucosylated steviol glycosides extract and present in a weight ratio of rebaudioside A extract to enzymatically glucosylated steviol glycosides extract that is from 1:4 to 5:1.
